(19) United States
(12) Patent Application Publication
(10) Pub. No.: US 2015/0257850 A1
(43) Pub. Date: Sep. 17, 2015

(54) IMAGE PROCESSING APPARATUS, IMAGE PROCESSING METHOD AND PROGRAM

(71) Applicant: TERUMO KABUSHIKI KAISHA, Tokyo (JP)

(72) Inventor: Masayuki SAKAMOTO, Hadano-City (JP)

(73) Assignee: TERUMO KABUSHIKI KAISHA, Tokyo (JP)

(21) Appl. No.: 14/657,283

(22) Filed: Mar. 13, 2015

(30) Foreign Application Priority Data
Mar. 14, 2014 (JP) 2014-052372

(51) Int. Cl. A61B 19/00; A61B 8/12; A61B 5/02; A61M 25/09; A61B 8/08; A61B 5/00; A61M 25/01

(52) U.S. Cl. CPC A61B 19/5244; A61B 5/0066; A61B 8/12; A61M 25/0108; A61M 25/09; A61B 5/0084; A61B 8/0841; A61B 8/0891; A61B 5/02007; A61B 8/5223; A61B 5/004; A61B 2019/5234; A61B 2019/528; A61B 2019/5265; A61B 2019/5274; A61B 2019/5287; A61M 2025/0166

(57) ABSTRACT

An image processing apparatus is disclosed, which obtains a tomographic image of a tubular body by scanning an inside of a first tubular body using a probe is acquired. Multiple points indicating an inner surface of the tubular body are detected on the tomographic image. Based on a position of the detected multiple points indicating the inner surface, at least one is determined between whether the point indicating the inner surface indicates a first tubular body and the point indicates a second tubular body bifurcated from the first tubular body, and whether the point indicating the inner surface indicates a boundary between the first tubular body and the second tubular body.

### CROSS-REFERENCES TO RELATED APPLICATIONS

This application claims priority to Japanese Application No. 2014-052372 filed on Mar. 14, 2014, the entire content of which is incorporated herein by reference.

### TECHNICAL FIELD

The disclosure generally relates to an image processing apparatus, an image processing method and a program.

### BACKGROUND DISCUSSION

In the related art, an apparatus is known which performs tomography by inserting a probe into a tubular body in order to perform image diagnosis on the tubular body such as a blood vessel. For example, an imaging apparatus for diagnosis has been used for diagnosis of arteriosclerosis, preoperative diagnosis in performing endovascular treatment using a high-performance catheter such as a balloon catheter or a stent, or for confirmation of postoperative results. For example, as a representative imaging apparatus for diagnosis, an intravascular ultrasound (IVUS) diagnosis apparatus and an optical coherence tomography/optical frequency-domain imaging (OCT/OFDI) diagnosis apparatus have been developed.

A technology has also been developed which can enable a doctor to relatively easily confirm a blood vessel image captured by these apparatuses. Japanese Patent Application Publication No. 2012-075702 discloses a technology in which a three-dimensional image reconstructed from a tomographic image obtained by IVUS or OCT is associated with a three-dimensional image obtained by CT or MRI. In the technology disclosed in Japanese Patent Application Publication No. 2012-075702, for the purpose of this association, a bifurcated portion of a blood vessel is automatically extracted from the image obtained by IVUS or OCT.

### SUMMARY

In some cases, it can be desirable to confirm not only information related to a probe-inserted tubular body but also information related to a bifurcated tube portion which is bifurcated from the tubular body. For example, when a stent is placed in a bifurcated portion of a blood vessel, it can be desirable to confirm not only information related to a main blood vessel but also information related to a bifurcated blood vessel, which is bifurcated from the main blood vessel. However, a technology for determining which portion within a tubular body image obtained by tomography indicates the probe-inserted tubular body and which portion indicates the bifurcated tube portion bifurcated from the tubular body has not yet been sufficiently developed.

The disclosure here generally aims to automatically determine a portion indicating an inner wall of a tubular body and a portion indicating an inner wall of a bifurcated tube portion bifurcated from the tubular body, or a boundary portion between the inner wall of the tubular body and the inner wall of the bifurcated tube portion, within an image obtained by performing tomography on the tubular body.

In accordance with an exemplary embodiment, an image processing apparatus is disclosed, which includes image acquisition means for acquiring a tomographic image of a tubular body which is obtained by scanning the inside of a first tubular body using a probe, detection means for detecting multiple points indicating an inner surface of the tubular body on the tomographic image, and determination means for determining at least either whether the point indicating the inner surface indicates the first tubular body and whether the point indicates a second tubular body bifurcated from the first tubular body, or whether the point indicating the inner surface indicates a boundary between the first tubular body and the second tubular body, based on a position of the detected multiple points indicating the inner surface.

In accordance with an exemplary embodiment, an image processing apparatus is disclosed, which can automatically determine a portion indicating an inner wall of a tubular body and a portion indicating an inner wall of a bifurcated tube portion bifurcated from the tubular body, or a boundary portion between the inner wall of the tubular body and the inner wall of the bifurcated tube portion, within an image obtained by performing tomography on the tubular body.

An image processing apparatus is disclosed, comprising: image acquisition means for acquiring a tomographic image of a tubular body which is obtained by scanning an inside of a first tubular body using a probe; detection means for detecting multiple points indicating an inner surface of the tubular body on the tomographic image; and determination means for determining at least either whether a point indicating the inner surface indicates the first tubular body and whether the point indicates a second tubular body bifurcated from the first tubular body, or whether the point indicating the inner surface indicates a boundary between the first tubular body and the second tubular body, based on a position of the detected multiple points indicating the inner surface.

An image processing method performed by an image processing apparatus is disclosed, comprising: an image acquisition step of acquiring a tomographic image of a tubular body which is obtained by scanning an inside of a first tubular body using a probe; a detection step of detecting multiple points indicating an inner surface of the tubular body on the tomographic image; and a determination step of determining at least one between whether a point indicating the inner surface indicates the first tubular body and the point indicates a second tubular body bifurcated from the first tubular body, and whether the point indicating the inner surface indicates a boundary between the first tubular body and the second tubular body, based on a position of the detected multiple points indicating the inner surface.

A non-transitory computer readable medium containing a computer program having computer readable code embodied to carry out an image processing method performed by an image processing apparatus is disclosed, the method comprising: an image acquisition step of acquiring a tomographic image of a tubular body which is obtained by scanning an inside of a first tubular body using a probe; a detection step of detecting multiple points indicating an inner surface of the tubular body on the tomographic image; and a determination step of determining at least one between whether a point indicating the inner surface indicates the first tubular body and the point indicates a second tubular body bifurcated from the first tubular body, and whether the point indicating the inner surface indicates a boundary between the first tubular body and the second tubular body, based on a position of the detected multiple points indicating the inner surface.

### BRIEF DESCRIPTION OF THE DRAWINGS

The accompanying drawings are incorporated in the description, configure a part of the description, represent embodiments of the image processing apparatus, and are used to describe principles of the image processing apparatus together with the description.

FIG. 1 is a diagram illustrating a schematic configuration of an image processing apparatus according to an exemplary embodiment of the present disclosure.

FIG. 2 is a diagram illustrating a schematic configuration of a boundary extraction unit according to an exemplary embodiment.

FIG. 3 is a flowchart of processing performed by the boundary extraction unit according to an exemplary embodiment.

FIGS. 4A-4F are views illustrating the processing performed by the boundary extraction unit according to an exemplary embodiment.

FIGS. 5A-5C are views illustrating processing performed by a determination update unit according to an exemplary embodiment.

FIGS. 6A-6C are views illustrating the processing performed by the determination update unit according to an exemplary embodiment.

FIGS. 7A-7C are views illustrating the processing performed by the determination update unit according to an exemplary embodiment.

FIGS. 8A-8B are views illustrating the processing performed by the determination update unit according to an exemplary embodiment.

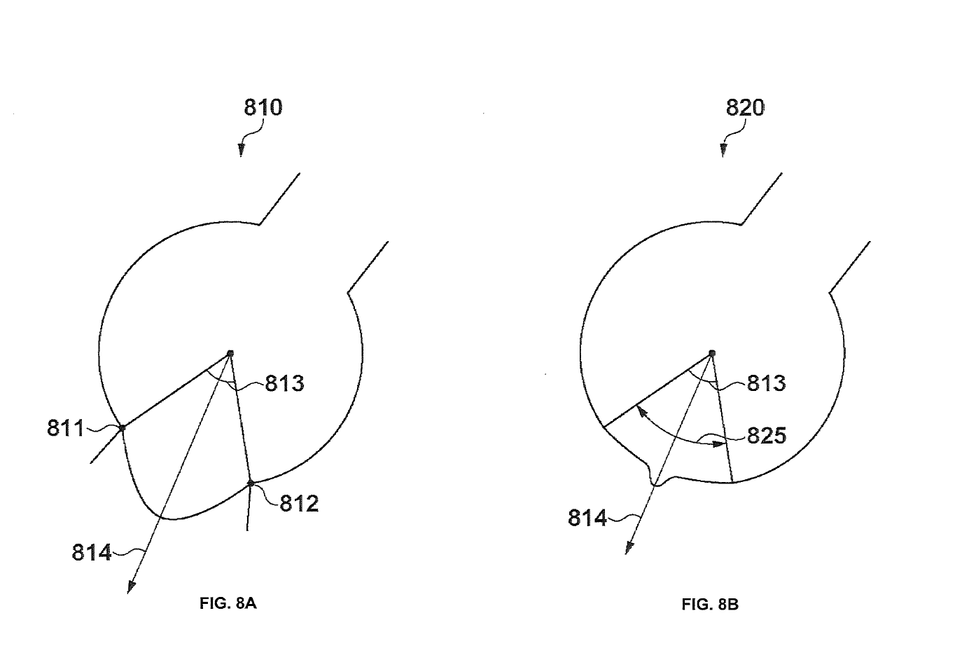

FIG. 9 is a view illustrating an example of a screen, which displays a determination result obtained by the boundary extraction unit.

FIG. 10 is a diagram illustrating a schematic configuration of an information calculation unit according to an exemplary embodiment.

FIG. 11 is a flowchart of processing performed by the information calculation unit according to an embodiment.

FIGS. 12A-12E are views illustrating an example of an approximation method for a boundary point group between a main blood vessel and a bifurcated blood vessel.

FIG. 13 is a view for illustrating an example of a definition of a bifurcated angle of the bifurcated blood vessel.

FIGS. 14A-14D are views illustrating a selection method for a point to be projected to a cross section of the bifurcated blood vessel.

FIG. 15 is a view illustrating an example of a display screen of information generated by the information calculation unit.

FIG. 16 is a diagram illustrating a basic configuration of a computer according to the embodiment.

### DETAILED DESCRIPTION

Hereinafter, an exemplary embodiment will be described with reference to the drawings. However, the scope of the disclosure is not limited to the exemplary embodiment described below.

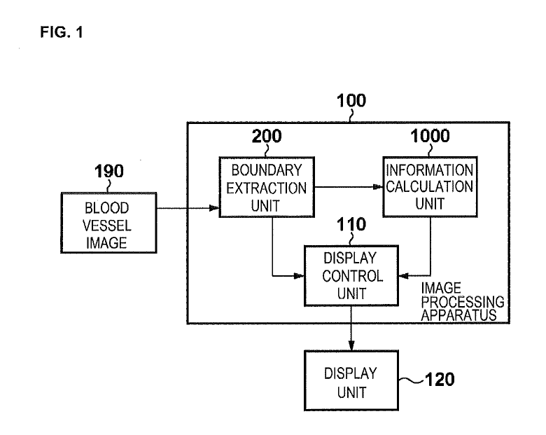

FIG. 1 illustrates an image processing apparatus 100 according to an exemplary embodiment. The image processing apparatus 100 can include a display control unit 110, a boundary extraction unit 200, and an information calculation unit 1000. The image processing apparatus 100 may be a part of an imaging system including an imaging device (not illustrated) which acquires a blood vessel image 190.

An image of a tubular body which is obtained by scanning the inside of the tubular body using a probe is input to the image processing apparatus 100. A type of the tubular body is not particularly limited. However, hereinafter, the tubular body will be a blood vessel and the blood vessel image 190 is input into the image processing apparatus 100.

The blood vessel image 190 can be image information indicating a form of the blood vessel, and the form is not particularly limited. An acquisition method of the blood vessel image is not particularly limited. For example, existing methods of an intravascular ultrasound (IVUS) diagnosis apparatus, an optical coherence tomography (OCT) diagnosis apparatus, or an optical frequency-domain imaging (OFDI) diagnosis apparatus can be used. Hereinafter, a probe-inserted blood vessel is called a main blood vessel.

The blood vessel image 190 can be acquired by inserting the probe into the blood vessel, and the inside of the blood vessel is scanned, thereby acquiring multiple line data items. Respective lines correspond to one scanning, and one line data item indicates a relationship between a distance from the probe to the blood vessel in a depth direction and obtained signal intensity. The multiple line data items can be obtained by performing scanning while changing an orientation of the probe and a position of the probe in a longitudinal direction of the blood vessel. In an exemplary embodiment, the blood vessel image 190 can be configured to include the multiple line data items. For example, the blood vessel image 190 may be configured so that one-dimensional images indicated by the line data items are laterally arrayed side by side. In addition, the blood vessel image 190 may be configured to include multiple cross-sectional images (images of cross-sections in a direction traversing a blood vessel axis, for example, images of cross-sections perpendicular to the blood vessel axis) obtained by circularly arraying the one-dimensional images indicated by the line data items. In addition, gain correction, contrast correction, or y correction may be performed on the blood vessel image 190. In the present exemplary embodiment, the blood vessel image 190 can be configured to include the multiple cross-sectional images whose gain and contrast are adjusted.

The blood vessel image 190 is not limited to a combination of the multiple cross-sectional images, and may be a combination of multiple longitudinal images (images of cross sections parallel to the blood vessel axis), or may be three-dimensional images of the blood vessel. As known, the cross-sectional images, the longitudinal images, and the three-dimensional images can be converted into one another.

In accordance with an exemplary embodiment, the boundary extraction unit 200 can detect a portion corresponding to the main blood vessel from the blood vessel image 190. In addition, the boundary extraction unit 200 can detect a portion corresponding to the bifurcated blood vessel bifurcated from the main blood vessel from the blood vessel image 190. In this manner, the boundary extraction unit 200 detects a boundary portion between the main blood vessel and the bifurcated blood vessel. In accordance with a detection result, the boundary extraction unit 200 can output information indicating the portion corresponding to the main blood vessel within the blood vessel image 190 and information distinguishing the portion corresponding to the bifurcated blood vessel to the information calculation unit 1000. However, as will be described later, it is not essential to detect the portion corresponding to the main blood vessel and the portion corresponding to the bifurcated blood vessel before detecting the boundary portion between the main blood vessel and the bifurcated blood vessel. A detailed configuration of the boundary extraction unit 200 will be described later. Here, the bifurcated blood vessel is a structure body connected to the main blood vessel into which the probe is inserted, and can include not only a tubular shape but also an aneurysm shape.

In accordance with an exemplary embodiment, the information calculation unit 1000 refers to the information obtained from the boundary extraction unit 200, and generates quantitative information indicating a form of the bifurcated blood vessel from the main blood vessel to the bifurcated portion. Then, the information calculation unit 1000 causes a display unit 120 to display the generated information via a display control unit 110.

The display control unit 110 controls the display unit 120 to display desired information. The display unit 120 is a device, which can display an image. The type of the display unit 120 is not particularly limited, and for example, may be a liquid crystal display.

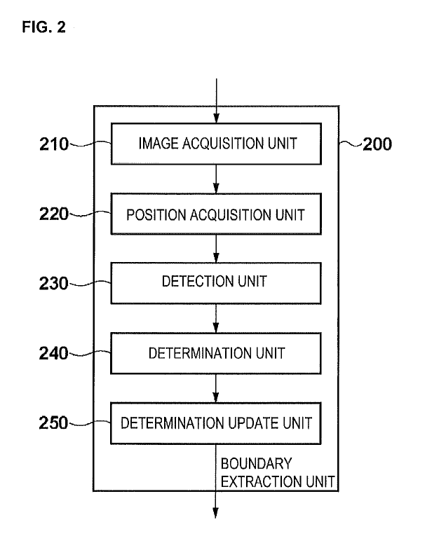

First, a schematic configuration of the boundary extraction unit 200 will be described with reference to FIG. 2. The boundary extraction unit 200 can include an image acquisition unit 210, a position acquisition unit 220, a detection unit 230, and a determination unit 240.

The image acquisition unit 210 acquires the blood vessel image 190. In the present exemplary embodiment, the image acquisition unit 210 acquires multiple tomographic images. Processing can be performed on the respective tomographic images by each unit (to be described later), thereby generating the information indicating the portion corresponding to the main blood vessel and the information distinguishing the portion corresponding to the bifurcated blood vessel. In addition, the image acquisition unit 210 can also perform pre-processing on the acquired tomographic image.

The position acquisition unit 220 acquires an estimated position of the center point of the main blood vessel on the tomographic image. In the present exemplary embodiment, the position acquisition unit 220 calculates the estimated position of the center point of the main blood vessel by performing image processing on the tomographic image. However, the center point of the main blood vessel may be designated by a user's input via an input unit (not illustrated).

In an exemplary embodiment, the position acquisition unit 220 acquires each estimated position of an image of a guidewire and a catheter sheath on the tomographic image. The position acquisition unit 220 calculates these estimated positions by performing the image processing on the tomographic image. However, these positions may be designated by a user's input via the input unit (not illustrated).

The detection unit 230 detects multiple points indicating an inner surface of the blood vessel on the tomographic image. As a detection method, various methods such as template matching can be considered. However, in the present exemplary embodiment, the detection unit 230 extracts a position of an intravascular wall on the tomographic image by scanning the intravascular wall to acquire the tomographic image. In accordance with an exemplary embodiment, for example, the detection unit 230 extracts an intersection point with the blood vessel when the intravascular wall proceeds outward from the center point of the main blood vessel, as the position of the intravascular wall.

The determination unit 240 can determine whether or not the multiple points detected by the extraction unit 230 indicate the main blood vessel. In addition, the determination unit 240 can determine whether or not the intersection point detected by the extraction unit 230 indicates the bifurcated blood vessel. In addition, the determination unit 240 can also determine whether each of the multiple points detected by the extraction unit 230 indicates the boundary between the main blood vessel and the bifurcated blood vessel. In accordance with an exemplary embodiment, these determinations can be performed based on the position of the multiple points, which can be detected by the extraction unit 230. In an exemplary embodiment, these determinations can be performed based on a positional relationship between the multiple continuous points, which can be detected by the extraction unit 230. For example, as will be described later, based on a change in the distance from the center of the main blood vessel with regard to the multiple continuous points, it can be possible to determine whether the point detected by the extraction unit 230 indicates the main blood vessel and indicates the bifurcated blood vessel. As will be described later, the determination on whether each point indicates the main blood vessel and indicates the bifurcated blood vessel based on a comparison between the distance from the center of the main blood vessel and a threshold value with regard to the multiple continuous points detected by the extraction unit 230 is also included in the determination based on the positional relationship between the multiple continuous points. In addition, it can be possible to determine that the bifurcated blood vessel is present in a portion where the multiple continuous points are disconnected from each other in the middle, and it can be possible to determine that an end of the multiple continuous points indicates the boundary between the main blood vessel and the bifurcated blood vessel.

In addition, these determinations can be made independently. For example, the determination unit 240 can determine at least either whether the point detected by the extraction unit 230 indicates the main blood vessel and indicates the bifurcated blood vessel, or whether the point detected by the extraction unit 230 indicates the boundary between the main blood vessel and the bifurcated blood vessel. The boundary between the main blood vessel and the bifurcated blood vessel can be detected by these determinations.

The distance between the center point and the intersection point of the main blood vessel can be mainly used in the determination using the determination unit 240. However, the determination unit 240 performs the determination by using other various references. For example, in an exemplary embodiment, the determination unit 240 performs the determination in view of the position of the image of the guidewire and the catheter sheath on the tomographic image.

In an exemplary embodiment, the boundary extraction unit 200 can further include a determination update unit 250. The determination update unit 250 updates a determination result obtained by the determination unit 240. For example, the determination update unit 250 can correct the determination result obtained by the determination unit 240. In addition, the determination update unit 250 can detect a new intersection point, and can determine that the detected intersection point indicates the main blood vessel or the bifurcated blood vessel. In addition, the determination update unit 250 can update the determination result obtained by the determination unit 240 with regard to another tomographic image, based on the determination result obtained by the determination unit 240 with regard to one tomographic image configuring the blood vessel image 190.

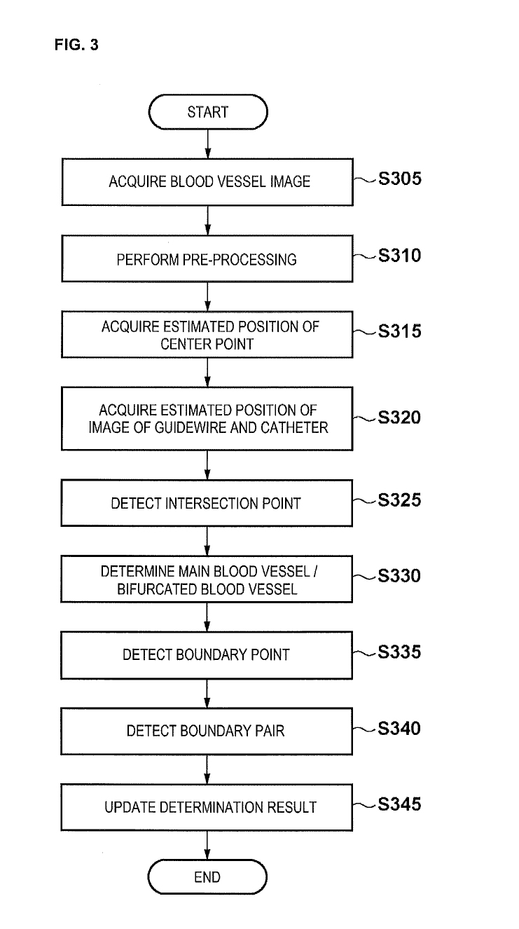

Next, referring to the flowchart in FIG. 3, processing performed by the boundary extraction unit 200 will be described in detail.

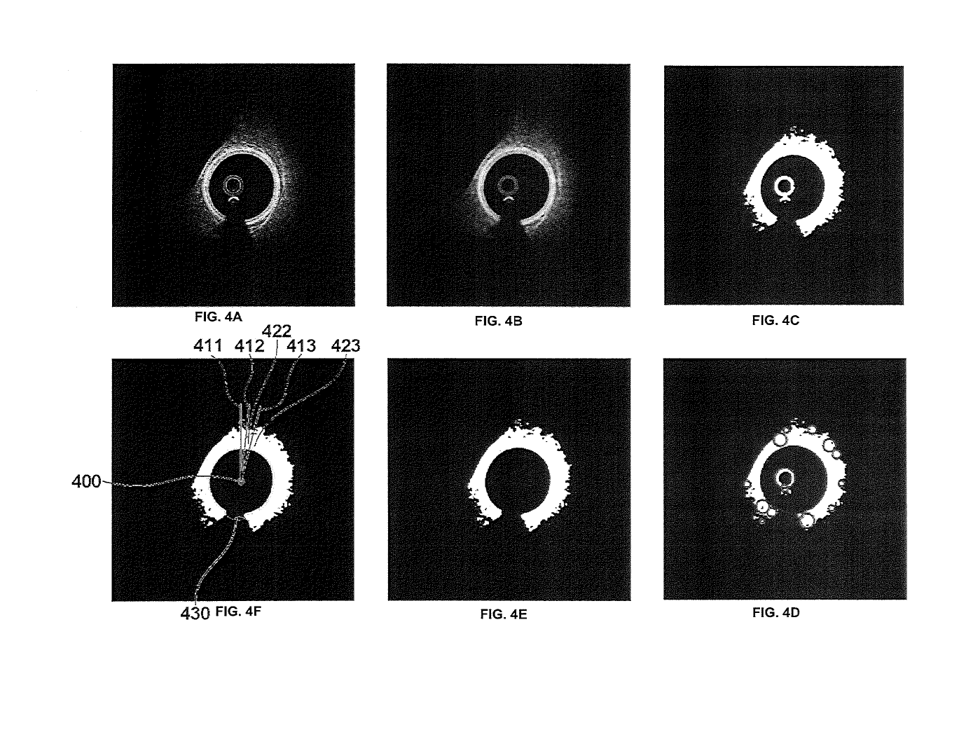

In Step S305, the image acquisition unit 210 acquires the blood vessel image 190. In the present exemplary embodiment, the image acquisition unit 210 is adapted to acquire the blood vessel image 190 configured to have multiple tomographic images at a time. However, the image acquisition unit 210 may sequentially acquire the tomographic images, which are sequentially generated while the blood vessel is scanned using the probe. The following processing subsequent to Step S310 is sequentially performed on each tomographic image. In the following description, FIG. 4A illustrates a tomographic image, which is a processing target.

In Step S310, the image acquisition unit 210 performs pre-processing on the acquired tomographic image. However, the image acquisition unit 210 may acquire a tomographic image on which the pre-processing has already been performed. A type of the pre-processing is not particularly limited. However, in one embodiment, filter processing and binary coded processing can be performed. A type of the filter processing to be used is not particularly limited. However, for example, smoothing filter processing can be used. In accordance with an exemplary embodiment, isolated points included as noise can be reduced by performing the smoothing filter processing on the tomographic image. FIG. 4B illustrates the tomographic image obtained after the filter processing.

In addition, processing (to be described later) can be simplified by performing binary coded processing using a predetermined threshold value. In the binary coded processing, a threshold value can be used so that an intravascular wall is indicated by a white pixel and an intravascular lumen is indicated by a black pixel. This threshold value may be set in advance, or may be automatically determined by the image acquisition unit 210 with reference to the histogram of the tomographic image. In addition, the threshold value may be input by a user via an input unit (not illustrated). Hereinafter, description will be made on the assumption that the binary coded processing has been performed in Step S310. However, it is not essential to perform the binary coded processing. When the binary coded processing is performed, it can be determined that a pixel having a pixel value inside a predetermined range defined by the threshold value is the white pixel, and it can be determined that a pixel having a pixel value outside the predetermined range is the black pixel. In addition, even when the binary coded processing is not performed, for example, in Step S325 (to be described later), the pixel having the pixel value inside the predetermined range is the white pixel can be determined, and the pixel having the pixel value outside the predetermined range is the black pixel can be determined. FIG. 4C illustrates the tomographic image subjected to the binary coded processing.

In Step S315, the position acquisition unit 220 acquires an estimated position of the center point of the main blood vessel on the tomographic image. As described above, the estimated position of the center point may be input by a user. However, in the present exemplary embodiment, the position acquisition unit 220 calculates the estimated position of the center point by performing image processing on the tomographic image.

A calculation method of the estimated position of the center point is not particularly limited. In an exemplary embodiment, the main blood vessel on the tomographic image can be detected, and the center of gravity can be used as the estimated position of the center point of the main blood vessel. In the present exemplary embodiment, as a detection method of the main blood vessel, the Hough transform can be used. In accordance with an exemplary embodiment, for example, the position acquisition unit 220 can detect a circle, which approximates a shape of the inner wall of the main blood vessel by performing the Hough transform processing on the tomographic image subjected to the binary coded processing. Then, the position acquisition unit 220 can acquire a center position of the detected circle as the estimated position of the center point. FIG. 4D illustrates an example of the circle detected by the Hough transform.

An estimation method of the center point using the Hough transform will be described in detail as follows. First, the position acquisition unit 220 extracts the circle of the black pixel (the circle in which the inner contour is indicated by the black pixel and the outer contour (background) is indicated by the white pixel) by performing the Hough transform. Then, the position acquisition unit 220 specifies the largest circle among the circles including the center of the tomographic image as the circle indicating the inner wall of the main blood vessel (hereinafter, referred to as a blood vessel circle). The center of this blood vessel circle is treated as the estimated position of the center point of the main blood vessel. In addition, the position acquisition unit 220 also calculates the radius of the blood vessel circle.

However, if the lumen of the main blood vessel does not have a substantially circular shape, there can be a possibility that the blood vessel circle cannot be extracted by performing the Hough transform. In this case, for example, the position acquisition unit 220 can employ the center of the blood vessel circle extracted from the tomographic image at another position of the main blood vessel, as the estimated position of the center point. The radius of the blood vessel circle is similarly calculated. In an exemplary embodiment, the center of the blood vessel circle extracted from the tomographic image at the closest position that is the tomographic image from which the blood vessel circle can be extracted by performing the Hough transform is used as the estimated position of the center point. In an exemplary embodiment, the estimated position of the center point in the tomographic image at the adjacent position can be used as the estimated position of the center point.

In accordance with an exemplary method, when the blood vessel circle cannot be extracted by performing the Hough transform, the position acquisition unit 220 may detect an inscribed circle of the blood vessel, and may treat the center of the inscribed circle as the estimated position of the center point of the main blood vessel. For example, on the tomographic image subjected to the binary coded processing, the position acquisition unit 220 can detect the circle, which can be inscribed in three or more white pixels and does not pass through the white pixel, as the inscribed circle of the blood vessel. In addition, for example, the largest circle among the circles inscribed in the blood vessel can be detected by using the Euclidian distance transform. The distance transform is not limited to the Euclidian distance transform, but weighting may be performed. In accordance with an exemplary embodiment, the center of gravity of the multiple points, which can be detected by the extraction unit 230 may be treated as the estimated position of the center point of the main blood vessel.

After the intersection point indicating the main blood vessel on the tomographic image is determined as will be described later, the position of the center of gravity in the intersection point indicating the main blood vessel can be calculated. In this case, the position acquisition unit 220 may employ the position of the center of gravity in the intersection point indicating the main blood vessel, which can be calculated with regard to the tomographic image at another position of the main blood vessel, as the estimated position of the center point. In accordance with an exemplary embodiment, when the blood vessel circle cannot be extracted by performing the Hough transform, the position acquisition unit 220 can calculate the position of the center of gravity in the intersection point indicating the main blood vessel with regard to the tomographic image at another position of the main blood vessel, as the estimated position of the center point.

When the center of the blood vessel circle extracted from the tomographic image at another position of the main blood vessel is employed as the estimated position of the center point in this way, there can be a possibility that the intersection point may be erroneously determined. Therefore, with regard to this tomographic image, the determination unit 240 may determine the intersection point, which becomes a wire shadow boundary pair with reference to the wire shadow boundary pair detected with regard to the adjacent tomographic image. For example, the determination unit 240 can determine that the wire shadow boundary pair is present in the same angular direction as the wire shadow boundary pair detected with regard to the adjacent tomographic image. In addition, the determination unit 240 can determine that the wire shadow boundary is present in the center in the respective angular directions of wire shadow boundaries detected with regard to two adjacent tomographic images.

In Step S320, the position acquisition unit 220 can further detect the estimated position of the image of the guidewire and the catheter sheath from the tomographic image. In general, for example, when the blood vessel image is captured, a guiding catheter (not illustrated) is inserted into the main blood vessel via the guidewire, and the probe covered with the catheter sheath is inserted into the guiding catheter. For this reason, the tomographic image can include the image of the guidewire and the catheter sheath, which are inserted into the main blood vessel. In the present exemplary embodiment, the determination unit 240 performs determination in view of the position of the image of the guidewire and the catheter sheath on the tomographic image.

In accordance with an exemplary embodiment, the position acquisition unit 220 can extract the circle of the white pixel (the circle in which the inner contour is indicated by the white pixel and the outer contour is indicated by the black pixel) by performing the Hough transform. Then, the position acquisition unit 220 can specify the circle closest to the center of the tomographic image as a circle indicating the catheter sheath (hereinafter, referred to as a sheath circle). The position acquisition unit 220 may treat only the circle inside the circle indicating the inner wall of the main blood vessel among the extracted circles, as a candidate of the circle indicating the catheter sheath. The center of this sheath circle can be treated as the estimated position of the catheter sheath image.

In addition, the position acquisition unit 220 can detect a pixel which is brightest among pixels present between the sheath circle and the blood vessel circle on the tomographic image prior to the binary coded processing. The detected pixel is treated as a pixel indicating a portion of the guidewire (hereinafter, referred to as a guidewire pixel), and the position of the guidewire pixel is treated as the estimated position of the guidewire image.

In Step S325, the detection unit 230 extracts the intersection point with the blood vessel when the intravascular wall proceeds outward from the center point of the main blood vessel, as the position of the intravascular wall. Hereinafter, specific processing thereof will be described.

First, the detection unit 230 performs pre-processing on the tomographic image subjected to the binary coded processing. For example, the detection unit 230 sets all pixels present within a predetermined distance from the center of the blood vessel circle, as the black pixel. The predetermined distance can be determined so as to be equal to or smaller than the radius of the blood vessel circle. This processing can decrease a possibility that the pixel, which is close to the center of the blood vessel circle and does not indicate the intravascular wall may be erroneously detected as the intravascular wall. In accordance with an exemplary embodiment, by causing the predetermined distance to be smaller than the radius of the blood vessel circle, it can be possible to decrease a possibility of failure in detecting the pixel indicating the intravascular wall, which is located near the blood vessel circle. The predetermined distance is not particularly limited. For example, the predetermined distance may be equal to or smaller than 0.95 times the radius of the blood vessel circle, or may be equal to or greater than 0.50 times the radius of the blood vessel circle.

For example, in accordance with an exemplary embodiment, this processing can erase the guidewire image, the catheter sheath image and noise of the intravascular lumen from the tomographic image. Therefore, the intersection point is no longer detected from a region in which the distance from the center point of the main blood vessel is equal to or smaller than the predetermined distance. In order to erase the guidewire image, the catheter sheath image and noise of the intravascular lumen, another exemplary method can be employed. For example, the guidewire image or the catheter sheath image can be recognized by using template matching, thereby enabling the recognized image to be erased. Furthermore, the noise or the catheter sheath image may be recognized and erased by using morphological processing. FIG. 4E illustrates the tomographic image immediately before the intersection point is detected.

Thereafter, the detection unit 230 performs scanning in each angular direction from the center point of the main blood vessel, thereby detecting the intersection point with the blood vessel, that is, the intersection point with the white pixel. In accordance with an exemplary embodiment, for example, with regard to respective multiple half-lines extending in each angular direction from the center point of the main blood vessel, the detection unit 230 detects the pixel closest to the center point among the white pixels on a half-line, as the intersection point corresponding to the angular direction. Scanning of 360° allows the pixel indicating the intravascular wall to be extracted. The detection unit 230 stores information by associating the angular direction and the distance from the center point of the main blood vessel with the respectively detected intersection points. By using the stored information, a graph can be created whose horizontal axis indicates a scanning angle and whose vertical axis indicates the distance from the center point of the main blood vessel.

FIG. 4F illustrates an example of the detection performed by the detection unit 230. In FIG. 4F, scanning is performed from a center 400 of the blood vessel circle in respective angular directions 411, 412, and 413. As a result, intersection points 421, 422, and 423 are detected.

In Step S330, the determination unit 240 determines whether the intersection point indicates the main blood vessel, based on the distance from the center point of the main blood vessel to the intersection point. In addition, the determination unit 240 determines whether the intersection point indicates the bifurcated blood vessel. In general, for example, the bifurcated blood vessel is separated from the main blood vessel on the tomographic image. Accordingly, it is considered that the inner wall of the bifurcated blood vessel is further separated from the center point of the main blood vessel as compared to the inner wall of the main blood vessel. Therefore, the determination unit 240 determines that the intersection point indicates the inner wall of the main blood vessel when the distance from the center point of the main blood vessel to the intersection point is equal to or smaller than a threshold value. The threshold value is determined based on the radius of the blood vessel circle. A specific determination method of the threshold value is not particularly limited. For example, the threshold value may be equal to or greater than 0.90 times the radius of the blood vessel circle, or may be equal to or smaller than 1.10 times the radius of the blood vessel circle.

In addition, in an exemplary embodiment, the determination unit 240 can determine that the intersection point indicates the inner wall of the bifurcated blood vessel when the distance from the center point of the main blood vessel to the intersection point is beyond the threshold value. However, the inner wall of the main blood vessel, which is located behind the guidewire when viewed from the probe, is not projected to the tomographic image. Then, there is a possibility that a non-projected region 430 illustrated in FIG. 4F (hereinafter, referred to as a shadow of the guidewire) may be erroneously recognized as the bifurcated blood vessel. Therefore, in the present exemplary embodiment, the determination unit 240 further takes the position of the catheter sheath image and the guidewire image into consideration, and determines whether the intersection point in which the distance from the center point of the main blood vessel is beyond the threshold value indicates the bifurcated blood vessel or indicates the shadow of the guidewire.

In accordance with an exemplary embodiment, for example, the determination unit 240 determines that the intersection point present near an extension line from the center of the sheath circle to the position of the guidewire pixel indicates the shadow of the guidewire, and that the intersection point absent near the extension line indicates the bifurcated blood vessel. For example, the determination unit 240 can determine that the intersection point indicates the shadow of the guidewire when the distance between the center point and the intersection point of the main blood vessel is beyond the threshold value, and when the intersection point is present within a predetermined angular range in a direction from the catheter sheath image toward the guidewire image on the tomographic image. In addition, the determination unit 240 can determine that the intersection point indicates the bifurcated blood vessel in which the distance between the center point and the intersection point of the main blood vessel is beyond the threshold value, and in which the intersection point is absent within a predetermined angular range in a direction from the catheter sheath image toward the guidewire image on the tomographic image. The angular range is not particularly limited, and can be appropriately set.

A determination method of the determination unit 240 is not limited to this method. For example, with regard to two intersection points located in the adjacent angular directions, the determination unit 240 can determine that the two intersection points indicate the shadow of the guidewire or the bifurcated blood vessel, when the distance from the center point to the intersection point of the main blood vessel is greatly changed. Then, the determination unit 240 can determine that the intersection point, which is not determined to indicate the shadow of the guidewire or the bifurcated blood vessel indicates the main blood vessel. For example, this method can be applied to the tomographic image from which the blood vessel circle cannot be detected by performing the Hough transform.

In Step S335, the determination unit 240 detects the intersection point indicating the boundary between the main blood vessel and the bifurcated blood vessel, in accordance with the determination result. In addition, the determination unit 240 detects the intersection point indicating the boundary between the main blood vessel and the shadow of the guidewire. In the present exemplary embodiment, with regard to the intersection points present in the adjacent angular directions, when one intersection point indicates the main blood vessel and the other intersection point indicates the bifurcated blood vessel, the determination unit 240 determines that the intersection point indicating the main blood vessel indicates the boundary between the main blood vessel and the bifurcated blood vessel. However, in this case, the determination unit 240 may determine that the intersection point indicating the bifurcated blood vessel indicates the boundary between the main blood vessel and the bifurcated blood vessel. If scanning performed by the detection unit 230 allows sufficiently high resolution in the angular direction, there is little difference in obtainable information regardless of which method is employed. In addition, with regard to the intersection points present in the adjacent angular directions, when one intersection point indicates the main blood vessel and the other intersection point indicates the shadow of the guidewire, the determination unit 240 determines that the intersection point indicating the main blood vessel indicates the boundary between the main blood vessel and the shadow of the guidewire. However, in this case, the determination unit 240 may determine that the intersection point indicating the shadow of the guidewire indicates the boundary between the main blood vessel and the shadow of the guidewire.

In addition, the intersection point indicating the bifurcated blood vessel or the shadow of the guidewire may not be detected in a portion where the inner wall of the main blood vessel is disconnected in the middle. For example, in a case illustrated in FIG. 4F, the intersection point indicating the shadow of the guidewire may not be detected in the angular direction where the shadow of the guidewire is located. In addition, depending on an angle formed between the inner wall of the main blood vessel and the inner wall of the bifurcated blood vessel, the intersection point indicating the bifurcated blood vessel may not be detected in the vicinity of the bifurcating position. Therefore, based on the position of the portion where the inner surface of the main blood vessel is disconnected in the middle on the tomographic image, the determination unit 240 can determine whether the point indicating the inner surface is located at the boundary between the main blood vessel and the bifurcated blood vessel (or the shadow of the guidewire).

In accordance with an exemplary embodiment, for example, with regard to the adjacent angular directions, when the intersection point indicating the main blood vessel is present in one direction but the intersection point indicating the main blood vessel is absent in the other direction, the determination unit 240 can determine that the intersection point indicates the boundary between the main blood vessel and the bifurcated blood vessel or the shadow of the guidewire (this intersection point is referred to as a boundary candidate point). For example, when the boundary candidate point is present within a predetermined angular range in a direction from the catheter sheath image toward the guidewire image, the determination unit 240 determines that the boundary candidate point indicates the boundary between the main blood vessel and the shadow of the guidewire. In addition, when the boundary candidate point is absent within the predetermined angular range in the direction from the catheter sheath image toward the guidewire image, the determination unit 240 determines that the boundary candidate point indicates the boundary between the main blood vessel and the bifurcated blood vessel. In accordance with an exemplary embodiment, this method can enable the determination unit 240 to determine whether multiple points detected by the extraction unit 230 respectively indicate the boundary between the main blood vessel and the bifurcated blood vessel. In the present exemplary embodiment, the determination unit 240 determines whether the point detected by the extraction unit 230 indicates the boundary between the main blood vessel and the bifurcated blood vessel after the determination on whether the point detected by the extraction unit 230 indicates the main blood vessel and indicates the bifurcated blood vessel. However, the determination unit 240 can determine whether the point detected by the extraction unit 230 indicates the boundary between the main blood vessel and the bifurcated blood vessel independently of the determination on whether the point detected by the extraction unit 230 indicates the main blood vessel and indicates the bifurcated blood vessel.

In Step S340, the determination unit 240 detects an intersection point pair, which interposes the bifurcated blood vessel portion therebetween and indicates the boundary between the main blood vessel and the bifurcated blood vessel. In accordance with an exemplary embodiment, for example, the determination unit 240 can detect a set of two intersection points which indicate the boundary between the main blood vessel and the bifurcated blood vessel and are present in two different angular directions, for example, a set of intersection points in which all of the intersection points present between the two angular directions indicate the bifurcated blood vessel (or the intersection point is absent). In the following description, a pair of the intersection points detected in this way is referred to as a bifurcated portion boundary pair. In addition, the determination unit 240 can detect a set of two intersection points which indicate the boundary between the main blood vessel and the shadow of the guidewire and are present in two different angular directions, for example, a set of intersection points in which all of the intersection points present between the two angular directions indicate the shadow of the guidewire (or the intersection point is absent). In the following description, a pair of the intersection points detected in this way is referred to as a wire shadow boundary pair. The bifurcated portion boundary pair and the wire shadow boundary pair, which are detected in this way, can be used for processing performed by the determination update unit 250.

In Step S345, the determination update unit 250 updates the determination result obtained by the determination unit 240 in Step S330. Here, the determination update unit 250 refers to at least any one of a position of the intersection point indicating the boundary between the main blood vessel and the bifurcated blood vessel and a position of the intersection point indicating the boundary between the main blood vessel and the shadow of the guidewire. For example, the determination update unit 250 can determine whether or not a parameter determined based on the position of the intersection point indicating the boundary therebetween meets a predetermined condition. When the parameter meets the predetermined condition, the determination update unit 250 can correct the determination result. In addition, the determination update unit 250 can detect a new intersection point based on the position of the intersection point indicating the boundary therebetween, and can perform determination on the detected intersection point. Hereinafter, with regard to an updating method of the determination result, five examples will be described. However, the updating method of the determination result may be considered to include various forms, and is not limited to the following examples.

### (1) Erroneous Detection of Crescent-Shaped Main Blood Vessel

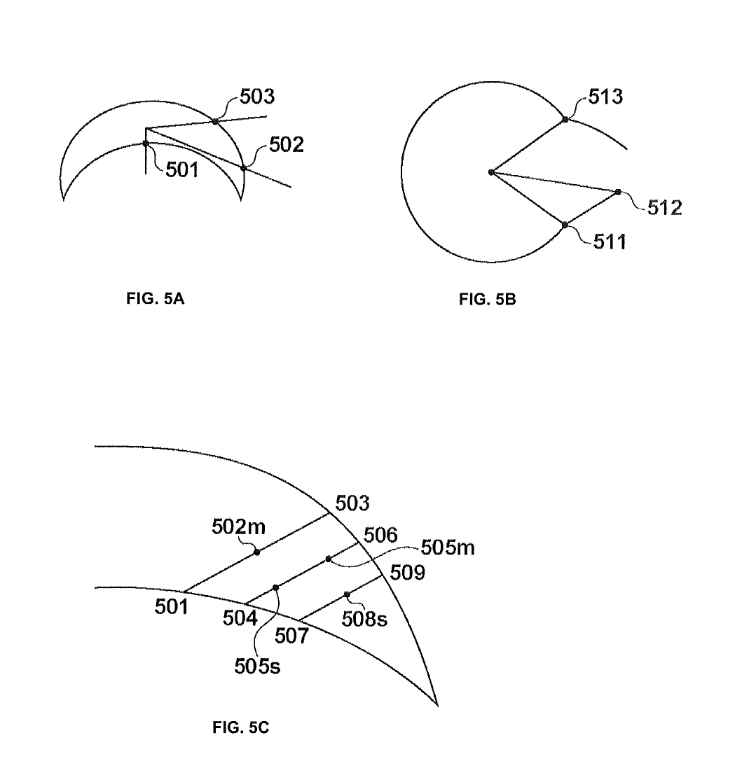

When the main blood vessel has a shape (crescent shape) illustrated in FIG. 5A, the intersection point may not be detected in a portion of the intravascular wall of the main blood vessel, or that the portion may be detected as the intersection point indicating the bifurcated blood vessel. For example, in a case of FIG. 5A, an intersection point 502 can be far away from the center point of the main blood vessel or the distance therefrom is greatly changed. Accordingly, the intersection point 502 may be detected as the intersection point indicating the bifurcated blood vessel. In this case, it can be considered that a difference increases between respective distances from the center point of the main blood vessel to a bifurcated portion boundary pair 501 and 503. For example, as illustrated in FIG. 5B, when the main blood vessel has a substantially circular shape and an intersection point 512 indicating the bifurcated blood vessel is detected, respective distances from the center point of the main blood vessel to a bifurcated portion boundary pair 511 and 513 are approximately the same as each other.

Therefore, in the present exemplary embodiment, the determination update unit 250 refers to the position of the intersection point indicating the boundary between the main blood vessel and the bifurcated blood vessel. When the reference meets a predetermined condition, the determination update unit 250 can determine that the intersection point determined to indicate the bifurcated blood vessel by the determination unit 240 indicates the main blood vessel. Then, the determination update unit 250 updates the determination performed by the determination unit 240. For example, when the difference between the respective distances from the center point of the main blood vessel to the bifurcated portion boundary pair is equal to or greater than a threshold value, the determination update unit 250 can determine that the intersection point determined to indicate the bifurcated blood vessel by the determination unit 240 indicates the main blood vessel. For example, in a case of FIG. 5A, the determination update unit 250 can determine that the intersection point 502 determined to indicate the bifurcated blood vessel by the determination unit 240 indicates the main blood vessel. In addition, the determination update unit 250 determines that the intersection points 501 and 503 are not the bifurcated portion boundary pair without indicating the boundary between the main blood vessel and the bifurcated blood vessel.

In accordance with an exemplary embodiment, when the main blood vessel has a shape illustrated in FIG. 5A, even if scanning is performed in each angular direction from the center point of the main blood vessel, the number of the intersection points which are detected near the intersection point 502 and indicate the main blood vessel decreases. Therefore, in the present exemplary embodiment, the determination update unit 250 additionally detects the intersection points which are present between the intersection points 501 and 503 determined not to be the bifurcated portion boundary pair and which indicate the main blood vessel. An example of the additional detection method will be described with reference to FIG. 5C. For example, the determination update unit 250 sets a straight line 505 obtained by causing a straight line 502 passing through the intersection points 501 and 503 determined not to be the bifurcated portion boundary pair to move in parallel by a predetermined distance in a direction away from the center point of the main blood vessel. Then, the determination update unit 250 detects intersection points 504 and 506 between the straight line 505 moved in parallel and the blood vessel, as the intersection point indicating the main blood vessel. In accordance with an exemplary embodiment, for example, the determination update unit 250 sets a point 505s obtained by causing a center point 502m of the intersection points 501 and 503 to move perpendicularly to the straight line 502 passing through the intersection points 501 and 503 by the predetermined distance in the direction away from the center point of the main blood vessel, as a reference point. Then, the determination update unit 250 performs scanning on the straight line 505 in both directions from the set reference point 505s, and detects a pair of the intersection points with the blood vessel, for example, the pair of the intersection points 504 and 506 with the white pixel.

In accordance with an exemplary embodiment, that detected intersection points 504 and 506, which are detected in this way may be the intersection points indicating the main blood vessel. Therefore, in view of a size of the blood vessel on the cross-sectional image on the upstream side or the downstream side, the determination update unit 250 determines whether or not the determination that the detected intersection points 504 and 506 indicate the main blood vessel is appropriate. The embodiment refers to the cross-sectional image on the upstream side or the downstream side, which has a substantially circular shape. For example, since the main blood vessel has the shape illustrated in FIG. 5A, this determination does not consider the cross-sectional image from which the intersection point indicating the main blood vessel is additionally detected. As an example of the determination method, the determination that the intersection points 504 and 506 are the intersection points indicating the main blood vessel is updated. In this manner, for example, when the updated size of the blood vessel is close to the approximate size of the blood vessel on the cross-sectional image on the upstream side or the downstream side, it is possible to determine that the determination is appropriate. When it is determined to be appropriate, the determination update unit 250 updates the determination performed by the determination unit 240, and determines that the intersection points 504 and 506 indicate the main blood vessel.

The determination update unit 250 can sequentially detect the intersection points indicating the main blood vessel by setting, the parallel movement, and scanning of a straight line passing through the pair of the intersection points indicating the main blood vessel which are newly detected in this way. For example, the determination update unit 250 sets a straight line 508 obtained by causing the straight line 505 passing through the intersection points 504 and 505 to move in parallel by the predetermined distance in the direction away from the center point of the main blood vessel. In addition, the determination update unit 250 sets a point 508s obtained by causing a center point 505m of the intersection points 504 and 506 to move perpendicularly to the straight line 505 passing through the intersection points 504 and 506 by the predetermined distance in the direction away from the center point of the main blood vessel, as a reference point. Then, the determination update unit 250 performs scanning on the straight line 508 in both directions from the set reference point 508s, and detects a pair of the intersection points with the blood vessel, that is, a pair of intersection points 507 and 509 with the white pixel. The determination update unit 250 determines that the detected intersection points 507 and 509 are the intersection points indicating the main blood vessel. If the reference point is the white pixel when this process is repeatedly performed, the repeated performance may be completed, or the distance for the parallel movement may be further shortened.

### (2) Erroneous Detection of Gourd-Shaped Main Blood Vessel

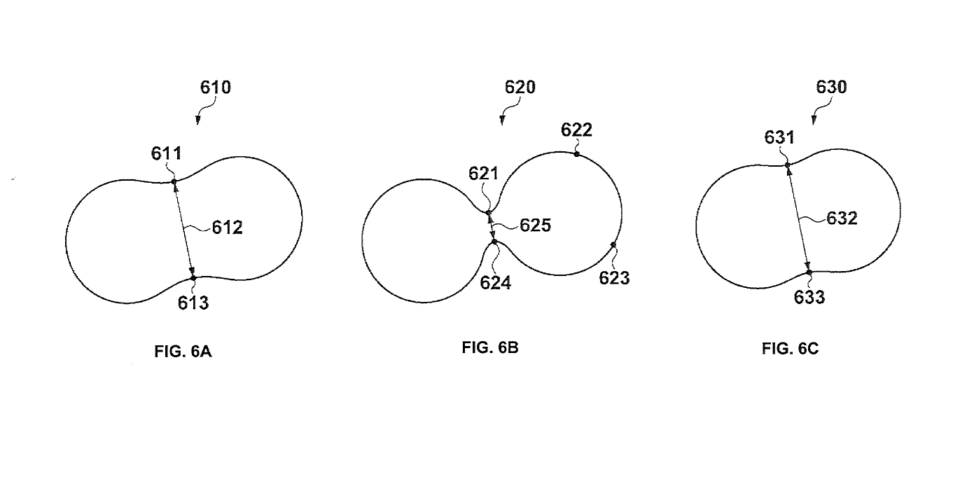

When the main blood vessel is narrowed and has a shape (gourd shape) illustrated in FIG. 6B, the intersection point may also not be detected in a portion of the intravascular wall of the main blood vessel, or that the portion may be detected as the intersection point indicating the bifurcated blood vessel. For example, in a case of a tomographic image 620 illustrated in FIG. 6B, intersection points 622 and 623 are far away from the center point of the main blood vessel. Accordingly, there is a possibility that the intersection points 622 and 623 may be detected as the intersection points indicating the bifurcated blood vessel. In this case, it can be determined that intersection points 621 and 624 are the bifurcated portion boundary pair.

As described above, the intersection point indicating the bifurcated blood vessel may be erroneously detected with regard to multiple tomographic images in the vicinity of the narrowed portion. If the preceding tomographic image is compared with the subsequent tomographic image in the narrowed portion, the main blood vessel is generally narrowed as it gets closer to the narrowed portion from the upstream side, and the main blood vessel is widened as it proceeds to the downstream side from the narrowed portion. FIG. 6B illustrates the tomographic image 620 in the most narrowed portion, and a reference numeral 625 represents a distance between a bifurcated portion boundary pair 621 and 624 on the tomographic image 620. FIG. 6A illustrates a tomographic image 610 located upstream from the tomographic image 620, and a reference numeral 612 represents a distance between a bifurcated portion boundary pair 611 and 613 on the tomographic image 610. In addition, FIG. 6C illustrates a tomographic image 630 located downstream from the tomographic image 620, and a reference numeral 632 represents a distance between a bifurcated portion boundary pair 631 and 633 on the tomographic image 630. As described above, the distance between the bifurcated portion boundary pair is shortened as it gets closer to the narrowed portion from the upstream side, and is lengthened as it proceeds to the downstream side from the narrowed portion. In accordance with an exemplary embodiment, for example, when the main blood vessel has a substantially circular shape and the bifurcated blood vessel is present, the distance between the bifurcated portion boundary pair is lengthened as it gets closer to the center of the bifurcated blood vessel from the upstream side, and is shortened as it gets closer to the downstream side from the center of the bifurcated blood vessel.

Therefore, in the present embodiment, the determination update unit 250 refers to a position of the intersection point indicating the boundary between the main blood vessel and the bifurcated blood vessel. When the reference meets a predetermined condition, the determination update unit 250 determines that the intersection point determined to indicate the bifurcated blood vessel by the determination unit 240 indicates the main blood vessel. In accordance with an exemplary embodiment, for example, the determination update unit 250 determines whether or not a distance 612 between the bifurcated portion boundary pair detected from the tomographic image 610 is longer than a distance 625 between the bifurcated portion boundary pair detected from the tomographic image 620. In addition, the determination update unit 250 determines whether or not a distance 632 between the bifurcated portion boundary pair detected from the tomographic image 630 is longer than the distance 625 between the bifurcated portion boundary pair detected from the tomographic image 620. When both of these conditions are met, there is a possibility that on the tomographic images 610, 620, and 630, the intersection point determined to indicate the bifurcated blood vessel by the determination unit 240 may indicate the main blood vessel. In addition, there is also a possibility that on the tomographic image between the tomographic image 610 and the tomographic image 630, the intersection point determined to indicate the bifurcated blood vessel by the determination unit 240 may indicate the main blood vessel.

Then, in view of a size of the blood vessel on the cross-sectional image on the upstream side or the downstream side, the determination update unit 250 determines whether or not the determination that the detected intersection points 622 and 623 indicate the main blood vessel is appropriate. This determination can be performed similarly to the determination with regard to the intersection points 504 and 506 described with reference to FIGS. 5A-5C. When it is determined as appropriate, if the intersection points 622 and 623 indicate the main blood vessel, the determination update unit 250 updates the determination performed by the determination unit 240. In addition, the determination update unit 250 determines that the intersection points 621 and 624 are not the bifurcated portion boundary pair without indicating the boundary between the main blood vessel and the bifurcated blood vessel.

When the main blood vessel has a shape illustrated in FIG. 6B, even if scanning is performed in each angular direction from the center point of the main blood vessel, the number of intersection points indicating the main blood vessel, which are detected near the intersection points 622 and 623, decreases. Therefore, in the present exemplary embodiment, the determination update unit 250 can additionally detect the intersection points which are present between the intersection points 621 and 624 determined not to be the bifurcated portion boundary pair and which indicate the main blood vessel. The additional detection can be performed similarly to a case where the main blood vessel has the shape illustrated in FIG. 5A.

(3) Erroneous Detection when Bifurcated Blood Vessel and Wire Shadow Overlap Each Other

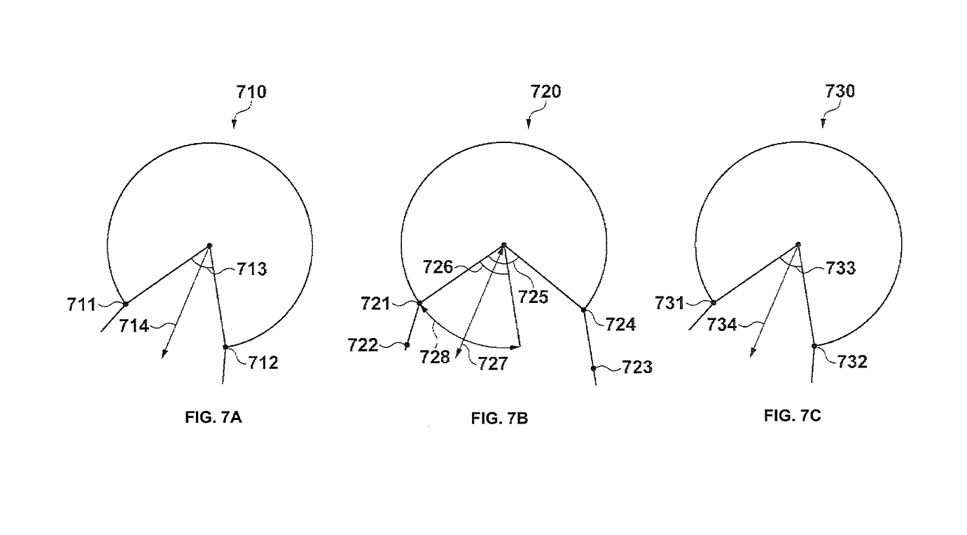

When the bifurcated blood vessel and the shadow of the guidewire overlap each other, depending on a position of the guidewire, there is a possibility of erroneous determination that the intersection point originally indicating the bifurcated blood vessel indicates the shadow of the guidewire. For example, on a tomographic image 720 illustrated in FIG. 7B, an intersection point 723 indicates the bifurcated blood vessel, but it is erroneously determined that the intersection point 723 indicates the shadow of the guidewire. In FIG. 7B, it is also determined that an intersection point 722 indicates the shadow of the guidewire, and it is determined that intersection points 721 and 724 are the wire shadow boundary pair. If this erroneous determination is performed, a size of the shadow of the guidewire indicated by the wire shadow boundary pair 721 and 724 increases.

In accordance with an exemplary embodiment, for example, on the tomographic images present at positions adjacent to each other, the sizes of the shadow of the guidewire are normally not too different from each other. FIG. 7A illustrates a tomographic image 710 at a position adjacent to a tomographic image 720 illustrated in FIG. 7B. In addition, FIG. 7C illustrates a tomographic image 730 at a position adjacent to the tomographic image 720 illustrated in FIG. 7B. On the tomographic image 710 and the tomographic image 730, the bifurcated blood vessel and the shadow of the guidewire do not overlap each other. In this case, for example, a size of the shadow of the guidewire indicated by a wire shadow boundary pair 711 and 712 is not significantly different from a size of the shadow of the guidewire indicated by a wire shadow boundary pair 731 and 732.

Therefore, the determination update unit 250 can refer to a position of the intersection point indicating the boundary between the main blood vessel and the shadow of the guidewire, and can determine that the intersection point determined to indicate the shadow of the guidewire by the determination unit 240 indicates the bifurcated blood vessel. In accordance with an exemplary embodiment, for example, when the size of the shadow of the guidewire is larger as compared to that of the tomographic image at the adjacent position, the determination update unit 250 can determine that the shadow of the guidewire and the bifurcated blood vessel overlap each other, and can determine that a portion within the intersection points determined to indicate the shadow of the guidewire indicates the bifurcated blood vessel.

Hereinafter, an example of a specific process performed by the determination update unit 250 will be described. First, the determination update unit 250 calculates an angular width and a central angle with regard to the detected wire shadow boundary pair. The angular width represents a difference between an angular direction from the center point of the main blood vessel to one of the wire shadow boundary pair and an angular direction from the center point of the main blood vessel to the other of the wire shadow boundary pair. In addition, the central angle represents an angular direction located in the center between the angular direction from the center point of the main blood vessel to one of the wire shadow boundary pair and the angular direction from the center point of the main blood vessel to the other of the wire shadow boundary pair. In FIGS. 7A and 7C, reference numerals 713 and 733 represent the angular width, and reference numerals 714 and 734 represent the central angle.

Then, the determination update unit 250 detects a tomographic image in which the angular width of the wire shadow boundary pair is wider than a threshold value. For example, in accordance with an exemplary embodiment, the determination update unit 250 can detect the tomographic image in which the angular width of the wire shadow boundary pair is beyond the threshold value. The threshold value may be set in advance, or may be calculated by the determination update unit 250. For example, this threshold value can be calculated based on an average value of the angular width calculated with regard to the predetermined number of tomographic images adjacent to the tomographic image serving as a detection target. In an example, the threshold value may be obtained by adding a predetermined value to the average value calculated in this way. In the following description, with regard to the tomographic image 720 illustrated in FIG. 7B, the determination update unit 250 can determine that an angular width 725 is beyond the threshold value.

If the tomographic image 720 is detected in which the angular width of the wire shadow boundary pair is beyond the threshold value, the determination update unit 250 estimates a portion where the shadow of the guidewire is located on this tomographic image 720, for example, a position of the wire shadow boundary pair. The determination update unit 250 can perform this estimation based on the position of the wire shadow boundary pair, which is calculated with regard to the tomographic images 710 and 730, located near the tomographic image serving as a processing target. In accordance with an exemplary embodiment, for example, the determination update unit 250 refers to the position of the wire shadow boundary pair 711 and 712 detected from the tomographic image 710 and the position of the wire shadow boundary pair 731 and 732 detected from the tomographic image 730, and estimates the position of the wire shadow boundary pair on a second tomographic image. Here, the tomographic image 710, the tomographic image 720, and the tomographic image 730 are respectively tomographic images located at first, second, and third positions of the blood vessel, and the second position is present between the first position and the third position.

In accordance with an exemplary embodiment, the determination update unit 250 can calculate a value by linear interpolation between the central angle 714 of the wire shadow boundary pair detected from the tomographic image 710 and the central angle 734 of the wire shadow boundary pair detected from the tomographic image 730. In this case, for example, the determination update unit 250 can estimate the calculated value as a central angle 727 of the wire shadow boundary pair with regard to the tomographic image 720. In addition, the determination update unit 250 can calculate a value by linear interpolation between the angular width 713 of the wire shadow boundary pair detected from the tomographic image 710 and the angular width 733 of the wire shadow boundary pair detected from the tomographic image 730. In this case, the determination update unit 250 can estimate the calculated value as an angular width 728 of the wire shadow boundary pair with regard to the tomographic image 720. Here, the linear interpolation may be weighted depending on a distance, or non-linear interpolation from three or more frames may be used.

Using the central angle 727 and the angular width 726 of the wire shadow boundary pair estimated in this way, the determination update unit 250 selects a portion of the intersection points determined to indicate the shadow of the guidewire on the tomographic image 720. In accordance with an exemplary embodiment, for example, the determination update unit 250 can select an intersection point 723 absent in a region 728 specified by the central angle 727 and the angular width 726 of the estimated wire shadow boundary pair, within the intersection points determined to indicate the shadow of the guidewire on the tomographic image 720. However, the determination update unit 250 may take a calculation error of the angular width 726 of the estimated wire shadow boundary pair into consideration, and may select the intersection point 723 after adding a predetermined angular value to the angular width 726. Then, the determination update unit 250 determines that the selected intersection point 723 indicates the bifurcated blood vessel. In addition, similarly to the determination unit 240, the determination update unit 250 can detect an intersection point 724 indicating a boundary point between the main blood vessel and the bifurcated blood vessel, in accordance with the new determination.

In order to accurately estimate the position of the wire shadow boundary pair, the determination update unit 250 can select a tomographic image in which the bifurcated blood vessel and the shadow of the guidewire do not overlap each other, as a tomographic image used for the estimation. For example, in an exemplary embodiment, the angular width 713 of the wire shadow boundary pair on the tomographic image 710 detected by the detection unit 230 is equal to or smaller than a threshold value, and the angular width 733 of the wire shadow boundary pair on the tomographic image 730 is equal to or smaller than a threshold value.

### (4) Additional Detection Near Bifurcating Start Portion of Bifurcated Blood Vessel

In accordance with an exemplary embodiment, the detection unit 230 can detect the intersection point with the blood vessel when the blood vessel proceeds in each angular direction from the center point of the main blood vessel in accordance with predetermined angular direction resolution. However, when the resolution is poor, there is a possibility that the bifurcated blood vessel cannot be extracted near a bifurcating start portion of the bifurcated blood vessel, for example, at a position away from the center of the bifurcated blood vessel. Therefore, in the present exemplary embodiment, the determination update unit 250 refers to the position of the intersection point indicating the boundary between the main blood vessel and the bifurcated blood vessel, and detects a portion having a possibility that the bifurcated blood vessel, which is not detected, may be present. Then, the determination update unit 250 detects the intersection point in accordance with higher angular direction resolution with regard to the detected portion, and determines whether the detected intersection point indicates the bifurcated blood vessel.

In accordance with an exemplary embodiment, the determination update unit 250 first detects a combination of a tomographic image 810 from which the bifurcated portion boundary pair is detected and a tomographic image 820 from which the bifurcated portion boundary pair is not detected. Here, the tomographic image 810 is a tomographic image present at the first position of the blood vessel, and the tomographic image 820 is a tomographic image present at the second position adjacent to the first position. The bifurcated blood vessel is projected to multiple tomographic images adjacent to each other. Accordingly, there is a possibility that the bifurcated blood vessel may not be detected from the tomographic image 820 but the bifurcated blood vessel may be projected thereto.

Next, the determination update unit 250 refers to an angular direction 813 of a bifurcated portion boundary pair 811 and 812 which is calculated from the tomographic image 810, and sets a detection range 825 on the tomographic image 820. This detection range 825 can be defined by a detection start angle and a detection end angle. For example, the detection range 825 can be set to include an angular direction 814. A size of the detection range 825 to be set is not particularly limited. In an exemplary embodiment, the size of the detection range 825 can be set to coincide with the angular width 813 of the bifurcated portion boundary pair 811 and 812. For example, the detection start angle coincides with an angular direction from the center point of the main blood vessel to the intersection point 811, and the detection end angle coincides with an angular direction from the center point of the main blood vessel to the intersection point 812. In an exemplary embodiment, in order to confirm a trend of a change in the distance from the center point of the main blood vessel to the intersection point, the size of the detection range 825 can be set to be larger than the angular width 813 of the bifurcated portion boundary pair 811 and 812 by a predetermined value. The angular width and the angular direction of the bifurcated portion boundary pair can be calculated similarly to the angular width and the angular direction of the wire shadow boundary pair.

Then, the determination update unit 250 detects the intersection point by using a more sensitive detection method, even while using the method similar to that of the detection unit 230 in the set detection range 825. Hereinafter, an example of improving sensitivity in detecting the intersection point will be described. However, the sensitivity improving method is not limited to the following example.

As an example, the determination update unit 250 can detect the intersection point in accordance with angular direction resolution higher than angular direction resolution when the detection unit 230 performs scanning. In one example, although the detection unit 230 performs the scanning for every one degree, the determination update unit 250 can perform the scanning for every 0.25 degrees. Then, similarly to the determination unit 240, the determination update unit 250 determines whether the detected intersection point indicates the main blood vessel or indicates the bifurcated blood vessel.

In addition, as described above, when the distance from the center point of the main blood vessel to the intersection point is significantly changed with regard to two intersection points located in the adjacent angular directions, the determination unit 240 can determine that the two intersection points indicate the bifurcated blood vessel (or the shadow of the guidewire). Using the same method, the determination update unit 250 can also determine that the intersection point indicates the bifurcated blood vessel. However, the determination update unit 250 can make a threshold value for the determination used in this case smaller than a threshold value used when the determination unit 240 performs the determination. For example, when a difference in distances from the center point of the main blood vessel to two intersection points is greater than a first threshold value, the determination unit 240 can determine that the two intersection points indicate the bifurcated blood vessel (or the shadow of the guidewire). In addition, when the difference in distances from the center point of the main blood vessel to two intersection points is greater than a second threshold value, the determination update unit 250 can determine that the two intersection points indicate the bifurcated blood vessel (or the shadow of the guidewire). Here, the second threshold value is smaller than the first threshold value.

In another method, with regard to the intersection points located in the adjacent angular directions, a trend of a change in the distance from the center point of the main blood vessel is detected, and it is determined that the intersection point in which the change in the distance is greater than the threshold value indicates the bifurcated blood vessel. For example, with regard to first, second, and third intersection points which are located in the adjacent angular directions, a difference is calculated between the difference in the distances from the center point of the main blood vessel to the first and second intersection points and the difference in the distances from the center point of the main blood vessel to the second and third intersection points. When the difference is greater than the first threshold value, the determination unit 240 can determine that the first intersection point indicates the bifurcated blood vessel (or the shadow of the guidewire). In addition, when the difference is greater than the second threshold value, the determination update unit 250 can determine that the first intersection point indicates the bifurcated blood vessel. Here, the second threshold value is also smaller than the first threshold value.

### (5) Additional Extraction of Bifurcated Blood Vessel

In a method used by the detection unit 230 for detecting the intersection point by performing scanning in each angular direction from the center point of the blood vessel, it is considered that the number of the intersection points detected in the bifurcated blood vessel portion decreases. Therefore, the determination update unit 250 refers to the position of the intersection point indicating the boundary between the main blood vessel and the bifurcated blood vessel, for example, the position of the bifurcated portion boundary pair. Then, the determination update unit 250 detects another intersection point indicating the bifurcated blood vessel, and determines that the detected intersection point indicates the bifurcated blood vessel.

The specific method is the same as the method of additionally detecting the intersection point indicating the main blood vessel, which has been described with reference to FIG. 5C. For example, the determination update unit 250 sets a straight line obtained by causing a straight line passing through the intersection points determined to be the bifurcated portion boundary pair to move in parallel by a predetermined distance in a direction away from the center point of the main blood vessel. Then, the determination update unit 250 detects the intersection point between the straight line moved in parallel and the blood vessel as the intersection point indicating the bifurcated blood vessel.

In an exemplary embodiment, the determination update unit 250 refers to the position of the intersection point indicating the boundary between the main blood vessel and the bifurcated blood vessel which has no bifurcated portion boundary pair formed therein. Then, the determination update unit 250 detects another intersection point indicating the bifurcated blood vessel, and determines that the detected intersection point indicates the bifurcated blood vessel. For example, as illustrated in FIG. 7B, when the bifurcated blood vessel and the shadow of the guidewire overlap each other, the intersection point can appear which has no bifurcated portion boundary pair formed therein and which indicates the boundary between the main blood vessel and the bifurcated blood vessel. In this case, the determination update unit 250 sets a straight line obtained by causing a straight line which passes through the intersection point 724 indicating the boundary between the main blood vessel and the bifurcated blood vessel and which is orthogonal to a direction indicated by the central angle 727 of the estimated wire shadow boundary pair to move in parallel by a predetermined distance in a direction away from the center point of the main blood vessel. Then, the determination update unit 250 can detect the intersection point between the straight line moved in parallel and the blood vessel as the intersection point indicating the bifurcated blood vessel.

In this case, the determination update unit 250 determines a point away from the intersection point 724 by a predetermined distance, along the straight line orthogonal to the direction indicated by the central angle 727, in a direction closer to the center point of the main blood vessel. Then, the determination update unit 250 sets a point obtained by causing the determined point to move in the direction indicated by the central angle 727 by a predetermined distance in a direction away from the center point of the main blood vessel, as a reference point. Furthermore, the determination update unit 250 performs scanning on a straight line set in a direction closer to the intersection point 724 from the set reference point, and can detect the intersection point with the blood vessel, that is, the intersection point with the white pixel, as the intersection point indicating the bifurcated blood vessel.

In an exemplary embodiment, instead of the intersection points determined to be the bifurcated portion boundary pair, the determination update unit 250 can use a pair of the intersection point 721 indicating the boundary between the main blood vessel and the shadow of the guidewire and the intersection point 724 indicating the boundary between the main blood vessel and the bifurcated blood vessel. For example, the determination update unit 250 sets a straight line obtained by causing a straight line passing through the pair to move in parallel by a predetermined distance in a direction away from the center point of the main blood vessel. Then, the determination update unit 250 detects the intersection point close to the intersection point 724 within the pair of intersection points between the straight line moved in parallel and the blood vessel, as the intersection point indicating the bifurcated blood vessel.

Using the same method, the determination update unit 250 may refer to the position of the wire shadow boundary pair, may detect another intersection point indicating the shadow of the guidewire, and may determine that the detected intersection point indicates the shadow of the guidewire.

The determination update unit 250 may use only one method among the above-described methods (1) to (5), or may use multiple methods in combination. In addition, a sequence for applying the above-described methods is not particularly limited. In an exemplary embodiment, in order to extract more intersection points indicating the bifurcated blood vessel, the determination update unit 250 performs the processing in (5) after performing the processes in methods (1) to (4). In the methods (2) to (4), the processing is performed by referring to at least any one of the position of the intersection point indicating the boundary between the main blood vessel and the bifurcated blood vessel and the position of the intersection point indicating the boundary between the main blood vessel and the shadow of the guidewire, on the tomographic image present at a position different from that of the tomographic image serving as a processing target. As described above, the boundary between the main blood vessel and the bifurcated blood vessel by referring to multiple tomographic images can be more accurately detected.

In accordance with an exemplary embodiment, the boundary extraction unit 200 sends the determination result obtained in this way to the information calculation unit 1000 in order to calculate quantitative information related to the bifurcated blood vessel. For example, positional information related to the respective intersection points and the determination result obtained by the determination unit 240 or the determination update unit 250 are sent to the information calculation unit 1000. In an exemplary embodiment, this positional information can include an angular direction from the center point of the main blood vessel and a distance from the center point. In this case, the boundary extraction unit 200 can send an estimated position of the center point of the main blood vessel on the tomographic image to the information calculation unit 1000. In an exemplary embodiment, this positional information may include XY coordinates of the intersection points on the tomographic image. As described above, the boundary extraction unit 200 generates the determination result with regard to multiple intersections for each tomographic image, and sends the determination result to the information calculation unit 1000.

However, it is not essential to send all of the determination results. The boundary extraction unit 200 sends information for distinguishing a portion corresponding to the main blood vessel from a portion corresponding to the bifurcated blood vessel bifurcated from the main blood vessel, to the information calculation unit 1000. For example, the boundary extraction unit 200 may send only the positional information related to the intersection point indicating the boundary between the main blood vessel and the bifurcated blood vessel, to the information calculation unit 1000. In addition, the boundary extraction unit 200 may send only the positional information related to the intersection point indicating the bifurcated blood vessel, to the information calculation unit 1000.

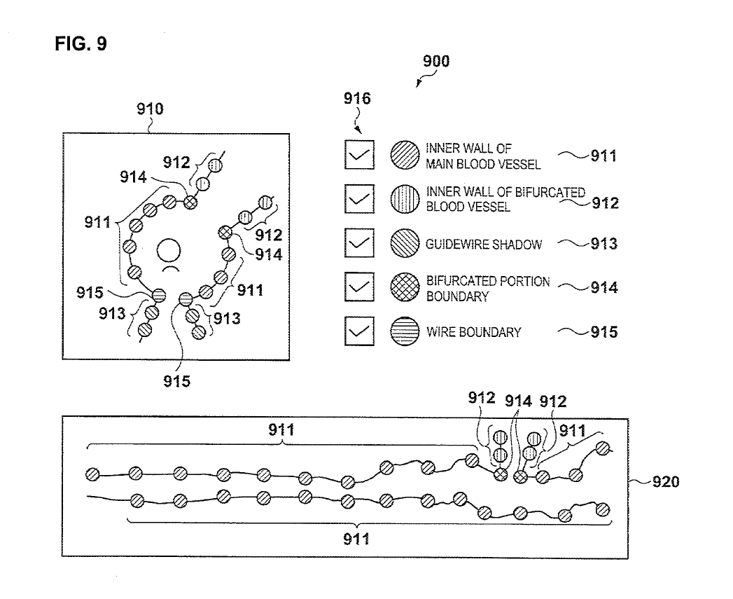

In addition, the boundary extraction unit 200 can cause the display unit 120 to display the determination result via the display control unit 110. FIG. 9 illustrates an example of a display screen. A cross-sectional image 910 and a longitudinal image 920 are displayed on a display screen 900 illustrated in FIG. 9. Dots 911 to 915, which indicate detected intersection points and are displayed in mutually different colors are displayed by being superimposed on one another on the cross-sectional image 910. The respective dots 911 to 915 display the intersection point indicating the main blood vessel, the intersection point indicating the bifurcated blood vessel, the intersection point indicating the shadow of the guidewire, the intersection point indicating the boundary point between the main blood vessel and the bifurcated blood vessel, and the intersection point indicating the boundary point between the main blood vessel and the shadow of the guidewire. For example, a user can stop display of these dots 911 to 915 by operating check boxes 916. Similarly, the dots 911 to 915 can also be displayed by being superimposed on one another on the longitudinal image. In this manner, the determination results automatically generated by the determination unit 240 or the determination update unit 250 can be displayed, thereby enabling a user to relatively easily recognize a portion corresponding to the bifurcated blood vessel or the shadow of the guidewire within the blood vessel image.

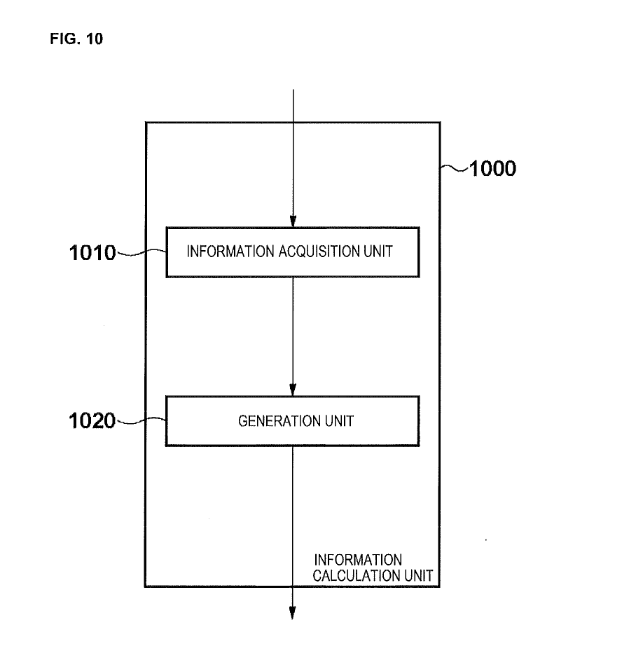

Next, a schematic configuration of the information calculation unit 1000 will be described with reference to FIG. 10. The information calculation unit 1000 includes an information acquisition unit 1010 and a generation unit 1020.

The information acquisition unit 1010 acquires information for distinguishing a portion corresponding to the main blood vessel from a portion corresponding to the bifurcated blood vessel bifurcated from the main blood vessel, within the blood vessel image 190. As described above, the information acquisition unit 1010 acquires the determination result obtained by the boundary extraction unit 200. In the present exemplary embodiment, the information acquisition unit 1010 acquires at least positional information related to the intersection point indicating the boundary between the main blood vessel and the bifurcated blood vessel or positional information related to the intersection point indicating the bifurcated blood vessel, from the boundary extraction unit 200. Furthermore, the information acquisition unit 1010 can acquire the blood vessel image 190.

The generation unit 1020 generates quantitative information indicating a form of the bifurcated blood vessel in the bifurcated portion from the main blood vessel by using the information acquired by the information acquisition unit 1010. For example, this quantitative information can include information indicating a size of the bifurcated blood vessel in the bifurcated portion from the main blood vessel and information indicating a bifurcating direction of the bifurcated blood vessel from the main blood vessel. In the present exemplary embodiment, the generation unit 1020 generates both the information indicating the size of the bifurcated blood vessel and the information indicating the bifurcating direction. However, the generation unit 1020 may generate any information between both of these.

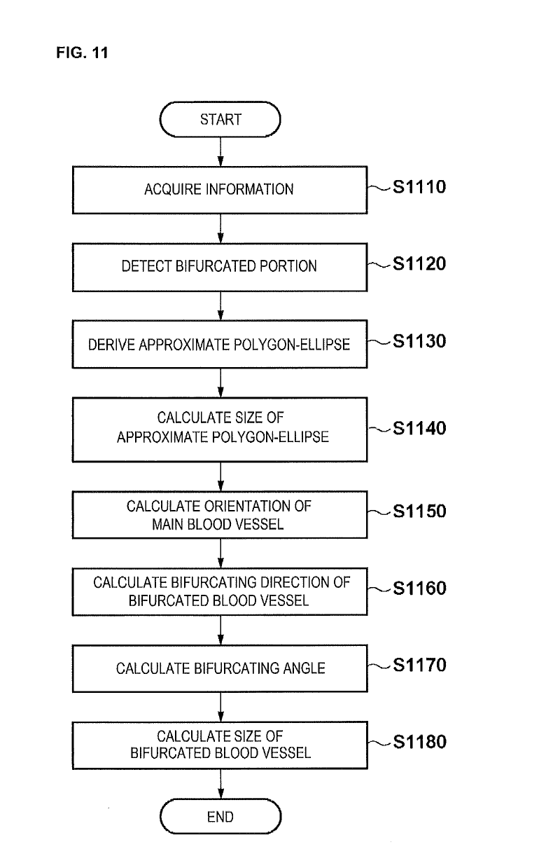

Next, referring to a flowchart in FIG. 11, processing performed by the information calculation unit 1000 will be described in detail.

In Step S1110, as described above, the information acquisition unit 1010 acquires the information for distinguishing the portion corresponding to the main blood vessel from the portion corresponding to the bifurcated blood vessel bifurcated from the main blood vessel, within the blood vessel image 190.

In Step S1120, the generation unit 1020 detects the bifurcated portion of the bifurcated blood vessel included in the blood vessel image 190. The generation unit 1020 determines that one bifurcated portion is present on the successive tomographic images on which the bifurcated blood vessel is present. It is possible to easily check whether the bifurcated blood vessel is present on the tomographic images by confirming the presence of the intersection point indicating the boundary between the main blood vessel and the bifurcated blood vessel or the presence of the intersection point indicating the bifurcated blood vessel. When multiple bifurcated portions are detected, the following process is performed on the respective bifurcated portions.

In Steps S1130 and S1140, the generation unit 1020 calculates bifurcated surface information related to the bifurcated portion detected in Step S1120. A bifurcated surface represents a surface connecting the main blood vessel and the bifurcated blood vessel to each other. An example of the bifurcated surface information can include a position, an orientation and a size of the bifurcated surface. The size of the bifurcated surface corresponds to the size of the bifurcated blood vessel in the bifurcated portion from the main blood vessel. The size of the bifurcated surface can include not only an area of the bifurcated surface but also a length indicating characteristics of the bifurcated surface (radius or diameter in case of a circle, and minor diameter or major diameter in case of an ellipse) and a circumferential length of the bifurcated surface.

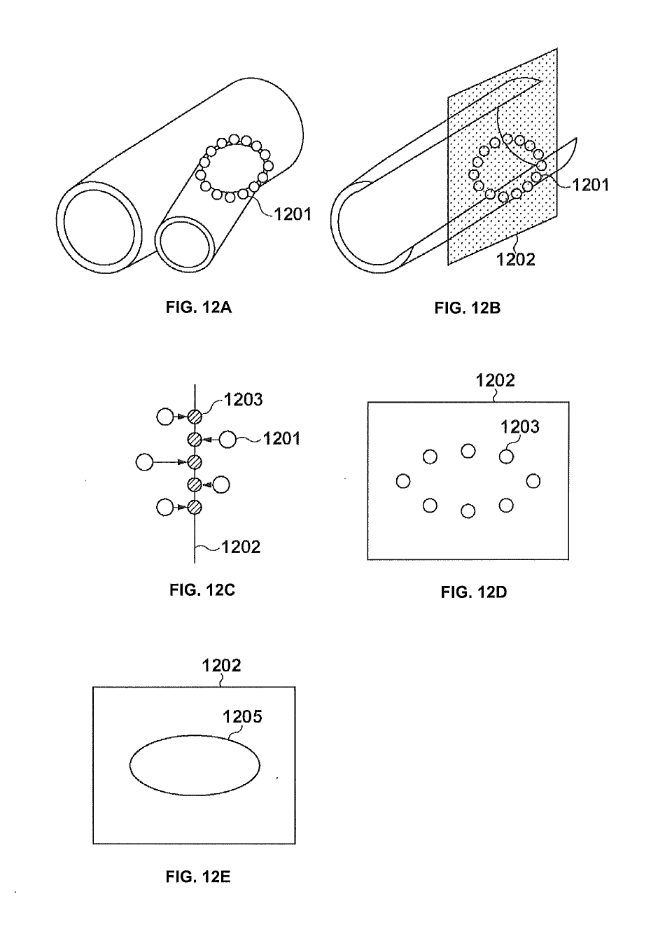

In Step S1130, the generation unit 1020 derives an approximate polygon or an approximate ellipse, which can approximate a boundary point group between the main blood vessel and the bifurcated blood vessel. For example, FIG. 12A illustrates a boundary point group 1201 which is an intersection point group indicating the boundary between the main blood vessel and the bifurcated blood vessel. In the present exemplary embodiment, as illustrated in FIG. 12B, the generation unit 1020 derives an approximate plane 1202, which can approximate the boundary point group 1201. Furthermore, as illustrated in FIG. 12C, the generation unit 1020 projects each boundary point group 1201 vertically to the approximate plane 1202, thereby obtaining a projection point group 1203. In this manner, as illustrated in FIG. 12D, an approximate polygon 1204 configured to have the projection point group 1203 is derived. The approximate polygon 1204 can be obtained by linearly connecting the adjacent projection point groups 1203 to each other. However, instead of the approximate polygon 1204, an approximate curve may be derived which is obtained by non-linearly connecting the projection point groups 1203 using a spline curve, for example. Then, as illustrated in FIG. 12E, the generation unit 1020 derives an approximate ellipse 1205, which can approximate the approximate polygon 1204. The algorithm used for the approximation is not particularly limited. For example, a known method can be employed, such as least square approximation.

In Step S1140, as quantitative information, the generation unit 1020 calculates a size of the derived approximate polygon (or the approximate curve), a size of the maximum inscribed circle of the derived approximate polygon (or the approximate curve), or a size of the derived approximate ellipse. For example, the generation unit 1020 can calculate an area of the derived approximate polygon. In addition, the generation unit 1020 can calculate a radius, a diameter, or an area of the maximum inscribed circle of the derived approximate polygon. Furthermore, the generation unit 1020 can calculate a minor axis length, a major axis length, or an area of the derived approximate ellipse. In addition, the generation unit 1020 can also calculate a circumferential length of the derived approximate polygon or the derived approximate ellipse. The generation unit 1020 may further calculate the center of the maximum inscribed circle of the derived approximate polygon, an intersection point between the major axis and the minor axis of the derived approximate ellipse, or the center of gravity of the derived approximate polygon or the derived approximate ellipse. Here, it is considered that the minor axis length of the approximate ellipse approximates the diameter of the bifurcated blood vessel in the bifurcated portion. In addition, it is considered that the area of the approximate polygon or the approximate ellipse approximates the area of the bifurcated surface.

Bifurcated surface information obtained in this way can be used as reference information for selecting devices such as a balloon and a stent, which are to be inserted into the bifurcated blood vessel. In Step S1130 and Step S1140, the generation unit 1020 may derive the maximum inscribed sphere inscribed in the boundary point group between the main blood vessel and the bifurcated blood vessel, and may calculate a size and a position of the derived maximum inscribed sphere. A value calculated in this way is also bifurcating information, and represents the size and the position of the bifurcated portion.

In Steps S1150 to S1170, the generation unit 1020 calculates a bifurcating direction of the bifurcated blood vessel from the main blood vessel with regard to the bifurcated portion detected in Step S1120.

In Step S1150, the generation unit 1020 calculates an orientation of the main blood vessel in the bifurcated portion. For example, the generation unit 1020 can calculate the center of gravity of the lumen of the main blood vessel on two or more tomographic images in the vicinity of the bifurcated portion, and can derive a vector, which approximates the position of the center of gravity. For this calculation, positional information related to the intersection point indicating the main blood vessel can be used, which can be obtained from the boundary extraction unit 200. The vector derived in this way represents the orientation of the main blood vessel. The center of gravity can be calculated by using the positional information related to the intersection point indicating the main blood vessel. The tomographic image used in calculating the orientation of the main blood vessel may be a tomographic image which has the bifurcated portion projected thereon, or may be a tomographic image present within a predetermined range from the bifurcated portion. However, instead of the center of gravity of the lumen of the main blood vessel, the center of the approximate ellipse of the lumen of the main blood vessel or the center of the maximum inscribed circle of the lumen of the main blood vessel can be used.

If a lesion is present in the vicinity of the bifurcated portion, the lumen of the main blood vessel is narrowed in the lesion, and thus, the position of the center of gravity is also moved. In one embodiment, if the lesion is present in the vicinity of the bifurcated portion, a tomographic image closer to the bifurcated portion than the lesion is used. In this case, it is possible to suppress influence of the lesion when the orientation of the main blood vessel is calculated. A detection method of the lesion is not particularly limited. However, for example, when the lumen of the main blood vessel is smaller than a threshold value, it is possible to determine that the lesion is projected to the tomographic image. In another embodiment, the generation unit 1020 specifies the tomographic image in which the maximum inscribed circle of the lumen of the main blood vessel is smallest, from the tomographic images having the lesion projected thereto. Then, instead of the center of gravity of the lumen of the main blood vessel, it is possible to use the center of the inscribed circle on the specified tomographic image. According to this configuration, the calculated bifurcating direction approximates an advancing direction of the bifurcated blood vessel with respect to an advancing direction of a catheter in the blood vessel. Accordingly, this configuration can be useful in making a treatment plan.

In Step S1160, the generation unit 1020 calculates the bifurcating direction of the bifurcated blood vessel in the bifurcated portion.

In the present exemplary embodiment, the generation unit 1020 calculates the bifurcating direction of the bifurcated blood vessel by referring to the positional information related to the intersection point indicating the bifurcated blood vessel. For example, the generation unit 1020 can derive an inscribed sphere, which is inscribed in the bifurcated blood vessel. Then, the generation unit 1020 can calculate the bifurcating direction of the bifurcated blood vessel, based on a direction from the center of gravity of the approximate polygon (or the approximate curve) or the approximate ellipse which is derived in Step S1140 toward the center of the derived inscribed sphere.

In accordance with an exemplary embodiment, the generation unit 1020 derives the maximum inscribed sphere, which is inscribed in the intersection point group indicating the bifurcated blood vessel. Here, the maximum inscribed sphere can be derived from a space surrounded by the intersection point group indicating the bifurcated blood vessel so as not to protrude to the bifurcated portion side or an open portion side (downstream side) away from the bifurcated portion. In addition, the maximum inscribed sphere may be derived after adding a point group indicating a start point of the bifurcated blood vessel and a point group on the open portion side (downstream side) away from the bifurcated portion to the intersection point group indicating the bifurcated blood vessel. Then, as a vector indicating the bifurcating direction of the bifurcated blood vessel, the generation unit 1020 calculates a vector oriented from the center of gravity of the approximate polygon (or the approximate curve) or the approximate ellipse which is calculated in Step S1140 toward the center of gravity of the maximum inscribed sphere. However, instead of the center of gravity of the approximate polygon (or the approximate curve) or the approximate ellipse, the generation unit 1020 may employ the center of the maximum inscribed circle of the approximate polygon (or the approximate curve) which is derived in Step S1140. In an exemplary embodiment, in order to improve calculation accuracy, the predetermined number of point groups away from the bifurcated portion is selected from the intersection point groups indicating the bifurcated blood vessel so as to derive the maximum inscribed sphere, which is inscribed in the selected point groups.

In an exemplary embodiment, the generation unit 1020 may derive two or more inscribed spheres which are inscribed in the intersection point group indicating the bifurcated blood vessel, and may calculate a direction from the center of one inscribed sphere toward the center of the other inscribed sphere, as the bifurcating direction of the bifurcated blood vessel. In this case, the bifurcating direction of the bifurcated blood vessel can be calculated based on only the positional information related to the intersection point indicating the bifurcated blood vessel. In an exemplary embodiment, in order to improve the calculation accuracy of the bifurcating direction of the bifurcated blood vessel, two inscribed spheres are derived so that one inscribed sphere is the maximum inscribed sphere, so that a distance between the centers of two inscribed spheres is equal to or greater than a threshold value, and so that a difference in radii of two inscribed spheres is equal to or smaller than a threshold value.

When a lesion is present in the bifurcated blood vessel, the processing can be performed, which is the same as that when a lesion is present in the main blood vessel. Using an angle, the generation unit 1020 can quantitatively indicate the bifurcating direction of the bifurcated blood vessel in the bifurcated portion obtained in this way. In an exemplary embodiment, the bifurcating direction can be indicated by an angle with respect to a scanning direction when the blood vessel image 190 is captured. The bifurcating direction obtained in this way can be used as reference information when a guidewire is inserted into the bifurcated blood vessel.

In accordance with an exemplary embodiment, for example, in Step S1170 according to the present embodiment, the generation unit 1020 calculates the bifurcating angle of the bifurcated blood vessel from the main blood vessel in order to provide more useful information. The bifurcating angle calculated in this way corresponds to quantitative information indicating a form of the bifurcated blood vessel in the bifurcated portion from the main blood vessel. The calculation method of the bifurcating angle is not particularly limited. For example, the generation unit 1020 may calculate an angle between a vector indicating the orientation of the main blood vessel and a vector indicating the bifurcating direction of the bifurcated blood vessel.

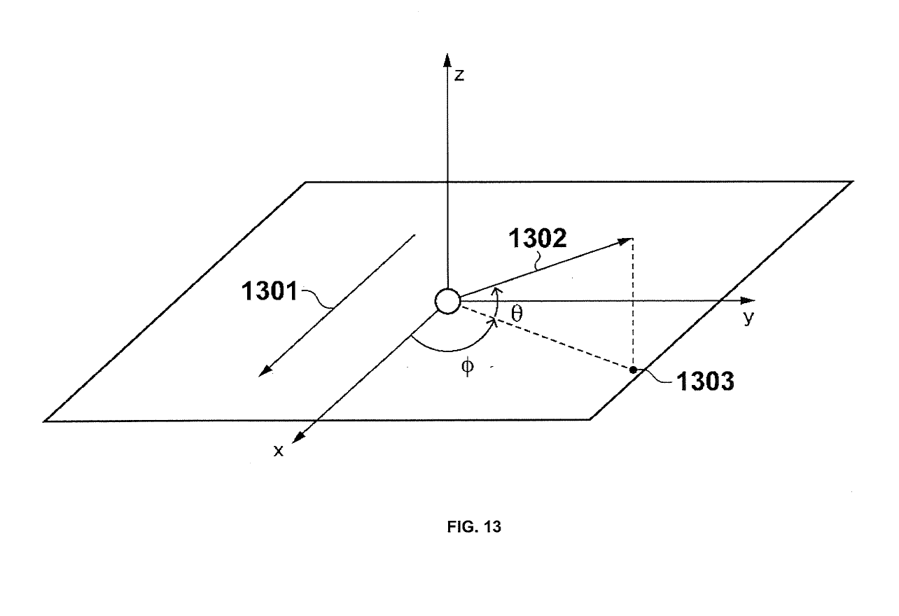

In an exemplary embodiment, in order for a user to easily and more intuitively understand the bifurcating angle, angles Φ and Θ illustrated in FIG. 13 are calculated. In FIG. 13, the xyz coordinate system (right-handed Cartesian coordinate system) is set so that a vector 1301 indicating the orientation of the main blood vessel is located on the xy plane and is parallel to the x-axis. In addition, the xyz coordinate system is set so that in a vector 1302 indicating the bifurcating direction of the bifurcated blood vessel, a start point is located at an original point and an end point is located in a first quadrant of the xy plane. In FIG. 13, a projection point 1303 is a point obtained by projecting the end point of the vector 1302 onto the xy plane.

The bifurcating angle obtained in this way can be used as reference information when a guidewire is inserted into the bifurcated blood vessel.

In Step S1180, the generation unit 1020 calculates information indicating a size of the bifurcated blood vessel on a cross section orthogonal to the bifurcating direction calculated in Step S1160. The information calculated in this way also corresponds to the quantitative information indicating the form of the bifurcated blood vessel in the bifurcated portion from the main blood vessel. For example, the information indicating the size of the bifurcated blood vessel can include a cross-sectional area, a circumferential length, or a radius or a diameter of the maximum inscribed circle. The generation unit 1020 can generate the information indicating the size of the bifurcated blood vessel in accordance with the intersection point group indicating the bifurcated blood vessel. In accordance with an exemplary embodiment, for example, a cross-sectional image of the bifurcated blood vessel can be generated by projecting a brightness of the intersection point group thereto. The brightness of the projected intersection point group has a brightness value of a pixel corresponding to the blood vessel image 190 (for example, the cross-sectional image).

For example, the generation unit 1020 may project the intersection point group indicating the bifurcated blood vessel to a cross section orthogonal to the bifurcating direction, or can calculate the information indicating the size of the bifurcated blood vessel in accordance with a position of the projected point. In this case, within the intersection point groups indicating the bifurcated blood vessel, a point group whose distance from a cross section is within a predetermined range can be projected to the cross section.

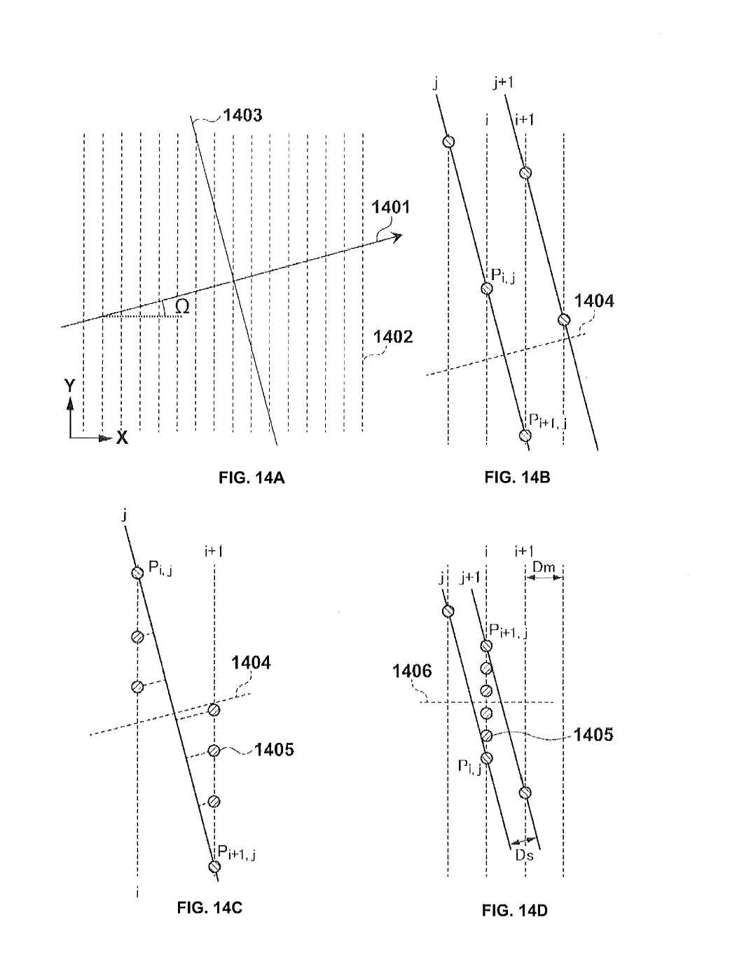

In an exemplary embodiment, the point group projected as follows is selected. This selection method will be described with reference to FIGS. 14A-14D. A vector 1401 indicating the bifurcating direction of the bifurcated blood vessel is set to be located on a plane, and the plane orthogonal to a projection cross section 1402 when the blood vessel image 190 is captured is set to be the XY plane. The X-axis represents a scanning direction when the blood vessel image 190 is captured. In addition, an angle formed between the scanning direction X and the vector indicating the bifurcating direction is set to Ω on the XY plane. Here, the i-th captured cross section when the blood vessel image 190 is captured is referred to as a cross section i, and a projection-targeted cross section 1403 orthogonal to the vector 1401 indicating the bifurcating direction is referred to as a cross section j. A relationship between both of these is illustrated in FIG. 14A.

First, with regard to each cross section i, a point is selected which is closest to the cross section j within the intersection point groups indicating the bifurcated blood vessel. The selected point is referred to as P_{i,j}. Next, when the points P_{i,j} and P_{i+1,j} are projected vertically to the XY plane, a vertically bisecting surface 1404 which bisects two projection points is calculated. FIG. 14B illustrates a position of the vertically bisecting surface 1404. Then, the intersection point group indicating the bifurcated blood vessel on the cross section i present between the vertically bisecting surface 1404 and a surface passing through P_{i,j} in parallel with the vertically bisecting surface 1404 is selected as a point to be projected to the cross section j. In addition, the intersection point group indicating the bifurcated blood vessel on a cross section i+1 present between the vertically bisecting surface 1404 and a surface passing through P_{i+1,j} in parallel with the vertically bisecting surface 1404 is selected as a point to be projected to the cross section j. FIG. 14C illustrates a point 1405 which is selected.

However, when multiple cross-sectional images are created, and when a distance Ds of the cross section j and a distance Dm of the cross section i satisfy a relationship of Ds<Dm·cos Ω, a portion of the intersection point groups indicating the bifurcated blood vessel is projected to both the cross section i and the cross section i+1. In order to avoid this case, the following processing may be performed. For example, when P_{i,j} and P_{i,j+1} are projected vertically to the XY plane, a vertically bisecting surface 1406 which bisects the two projection points is calculated. Then, the intersection point group indicating the bifurcated blood vessel on the cross section i present between the vertically bisecting surface 1406 and a surface passing through P_{i,j} in parallel with the vertically bisecting surface 1406 is projected to the cross section j, but is selected as a point which is not projected to a cross section j+1. In addition, the intersection point group indicating the bifurcated blood vessel on the cross section i present between the vertically bisecting surface 1406 and a surface passing through P_{i,j+1} in parallel with the vertically bisecting surface 1406 is projected to the cross section j+1, but is selected as a point which is not projected to the cross section j.

The creation method of the tomographic image on any cross section from the blood vessel image 190 is known as a multi-planar reconstruction method. Therefore, a method of creating the tomographic image on a cross section orthogonal to the bifurcating direction of the bifurcated blood vessel by reconstructing the blood vessel image 190 by the generation unit 1020 is not limited to the above-described method.

In accordance with an exemplary embodiment, an example of the calculation method of the information indicating the size of the bifurcated blood vessel on the cross section orthogonal to the bifurcating direction can include a method of obtaining an area or a circumferential length by deriving the approximate ellipse, the approximate curve and the approximate polygon which approximate the projected points, and a method of obtaining a radius, a diameter, or an area by deriving the maximum inscribed circle of the projected points.

As described above, the generation unit 1020 can create and output the tomographic image on the cross section orthogonal to the bifurcating direction of the bifurcated blood vessel. For example, the generation unit 1020 can create the tomographic image passing through the center of gravity of the approximate polygon (or the approximate curve) or the approximate ellipse which is calculated in Step S1140. In addition, the generation unit 1020 can also create the tomographic image passing through the most upstream side position or the most downstream side position within the bifurcated surface. The generation unit 1020 can generate a surface rendering image or a volume rendering image of the bifurcated blood vessel in a viewing direction along the bifurcating direction of the bifurcated blood vessel or in a viewing direction orthogonal to the bifurcating direction.

According to this configuration, a narrowed degree of the bifurcated blood vessel can be easily confirmed by moving a plaque in the bifurcated blood vessel direction after treatment is performed on the main blood vessel using a balloon or a stent. In addition, according to the above-described method, for example, even when the intersection point indicating the bifurcated blood vessel cannot be sufficiently detected on some tomographic images since the bifurcated blood vessel and the shadow of the guidewire overlap each other, quantitative information indicating a form of the bifurcated blood vessel can be obtained by using some intersection points.

When a stent is placed in the bifurcated portion, in Step S1140, the generation unit 1020 may calculate a size of a stent strut (column of the stent), for example, a width or a thickness. The stent strut appears as a very bright point on the tomographic image. Therefore, similarly to an estimated position of the guidewire image, the detection unit 230 can morphologically extract the stent strut as an object, and can detect the position. In this case, the generation unit 1020 can project the object representing the stent strut vertically to the bifurcated surface. Then, the generation unit 1020 can calculate an area in a divided region generated by dividing the bifurcated surface using the stent strut, or a size of the maximum inscribed circle. The information obtained in this way enables a user to confirm the width or the thickness of the stent strut after the stent is arranged in the main blood vessel.

In addition, in Step S1180, the generation unit 1020 can project the object representing the stent strut to the cross section orthogonal to the bifurcating direction of the bifurcated blood vessel. Then, on the cross section, the generation unit 1020 can calculate the area and the circumferential length of the divided region divided by the stent strut, or the size of the maximum inscribed circle. The information obtained in this way corresponds to the width of the stent strut in a direction vertical to a catheter when the catheter is operated so as to pass through a gap of the stent strut. Therefore, this information is useful when a guidewire is operated so as to pass through the gap of the stent strut, for example, in order to select a balloon used in performing a kissing balloon technique.

Furthermore, when the stent is arranged in the bifurcated portion, the generation unit 1020 can correct the quantitative information indicating the form of the bifurcated blood vessel in the bifurcated portion from the main blood vessel by using information indicating the size of the stent. The information indicating the size of the stent can be acquired by the information acquisition unit 1010. The information indicating the size of the stent may be input to an input unit (not illustrated) by a user, or may be recorded on the information calculation unit 1000 in advance.

For example, the information acquisition unit 1010 can acquire information indicating the width or the thickness (I1) of the stent strut. In this case, the generation unit 1020 can calculate a correction parameter (I1/I2) by using the calculated width or the calculated thickness (I2) of the stent strut. The width or the thickness of the stent strut which is to be calculated may be an average value of values measured for each of multiple stent struts. The generation unit 1020 can obtain a more accurate value by multiplying the correction parameter (I1/I2) calculated in this way and the quantitative information indicating the form of the bifurcated blood vessel, for example, the minor axis length of the approximate ellipse which is calculated in Step S1140. For example, when the width of the stent strut is used, the length in the axial direction of the blood vessel can be corrected. In addition, when the thickness of the stent strut is used, the length in the scanning line direction can be corrected.

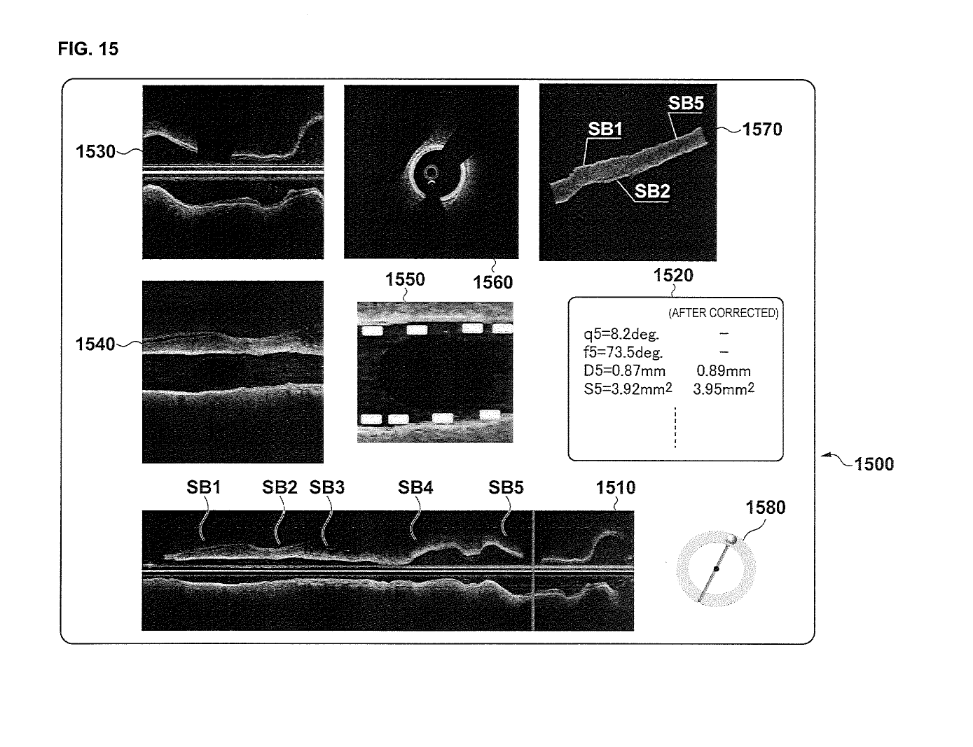

The information calculation unit 1000 can cause the display unit 120 to display the obtained quantitative information indicating the form of the bifurcated blood vessel via the display control unit 110. For example, the display control unit 110 can cause the display unit 120 to display a display screen including the quantitative information. In addition, the display control unit 110 can cause the display unit 120 to display the display screen, which can include at least one of the cross-sectional image, the longitudinal image, and the three-dimensional image of the blood vessel. FIG. 15 illustrates an example of the display screen, which can include both the quantitative information and the blood vessel image. However, the display screen may not necessarily include both the quantitative information and the blood vessel image.

A longitudinal image 1510 is displayed on a display screen 1500, and bifurcated portions SB1 to SB5 are displayed from the detected main blood vessel to the bifurcated blood vessel on the longitudinal image 1510. Via an input unit (not illustrated), a user can designate the bifurcated portion about which the user wants to know more information. The display control unit 110 can update the display screen in accordance with an input designating one of the multiple bifurcated portions so that the display screen includes quantitative information indicating a form of the bifurcated blood vessel in the designated bifurcated portion. In addition, the display control unit 110 can update the display screen in accordance with the input for designating one of the multiple bifurcated portions so that the display screen includes at least one of the cross-sectional image, the longitudinal image, and the three-dimensional image of the blood vessel including the designated bifurcated portion.

For example, the bifurcated portion SB5 can be selected on the display screen 1500. Bifurcating angles (Θ5 and Φ5), a diameter (D5) of the bifurcated blood vessel, and an area (S5) of the bifurcated surface are displayed in a region 1520 on the display screen 1500 illustrated in FIG. 15. In addition, the diameter (D5) of the bifurcated blood vessel and the area (S5) of the bifurcated surface which have been corrected by the width or the thickness of the stent strut are further displayed on the region 1520.

In addition, enlarged images 1530, 1540, and 1550 of the longitudinal image including the bifurcated portion SB5, and a cross-sectional image 1560 including the bifurcated portion SB5 are displayed on the display screen 1500. A cross-sectional position of the enlarged image 1530 to be displayed can be changed in a direction toward the depth of the screen. In addition, a cross-sectional orientation of the enlarged image 1530 can be controlled by using a user interface 1580 located at the bottom right of the display screen 1500. The enlarged image 1540 is a blood vessel axial direction cross section which passes through the center (probe) in parallel with the xz plane illustrated in FIG. 13. A position of the detected stent strut is highlighted and displayed on the enlarged image 1550. The longitudinal image and the cross-sectional image with regard to the main blood vessel are displayed on the display screen 1500. However, the longitudinal image or the cross-sectional image with regard to the bifurcated blood vessel in the bifurcated portion SB5 may be displayed thereon.

The three-dimensional image of the blood vessel including the bifurcated portion SB5 is displayed on a 3D image 1570. In one embodiment, a viewing direction of the displayed three-dimensional image is determined according to the bifurcating direction of the bifurcated blood vessel in the designated bifurcated portion. For example, the display control unit 110 can determine the viewing direction of the 3D image 1570 so that the viewing direction coincides with a vector of the bifurcating direction of the bifurcated blood vessel in the selected bifurcated portion SB5. The determination method of the viewing direction is not limited to this method. For example, the display control unit 110 may determine the viewing direction of the 3D image 1570 so that the viewing direction is orthogonal to the vector of the bifurcating direction of the bifurcated blood vessel in the selected bifurcated portion SB5.

Hitherto, the image processing apparatus 100 including the boundary extraction unit 200 and the information calculation unit 1000 has been described. However, an image processing apparatus including the boundary extraction unit 200 and an image processing apparatus including the information calculation unit 1000 may be respectively separate apparatuses. For example, in this case, the image processing apparatus including the boundary extraction unit 200 can acquire the blood vessel image 190, and can output the determination result obtained by the determination unit 240 or the determination update unit 250. Based on the output information, a user can easily recognize a portion corresponding to the bifurcated blood vessel or the shadow of the guidewire within the blood vessel image 190. In addition, the image processing apparatus including the information calculation unit 1000 may acquire information which is generated by a method different from the method employed by the boundary extraction unit 200, and which distinguishes a portion corresponding to the main blood vessel from a portion corresponding to the bifurcated blood vessel. Even when using this information, the image processing apparatus including the information calculation unit 1000 can generate and output quantitative information indicating the obtained form of the bifurcated blood vessel.

A function of each unit included in the image processing apparatus 100 illustrated in FIG. 1 can be realized by using a general-purpose computer. As described above, even when the image processing apparatus including the boundary extraction unit 200 and the image processing apparatus including the information calculation unit 1000 are respectively separate apparatuses, a function of the respective apparatuses can be realized by using the general-purpose computer.

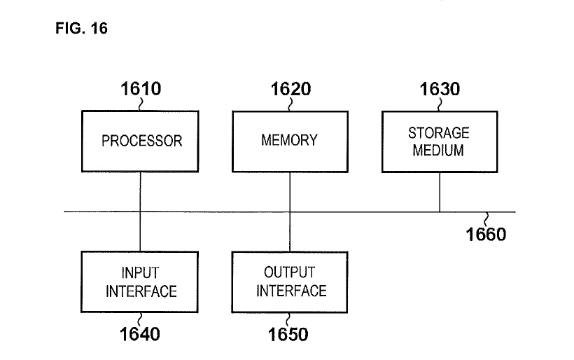

FIG. 16 is a view illustrating a basic configuration of the computer. For example, a processor 1610 in FIG. 16 is a CPU, and controls overall operations of the computer. For example, a memory 1620 is a RAM, and temporarily stores a program and data. For example, a computer-readable storage medium 1630 is a hard disc or a CD-ROM, and stores the program and the data for a long time. In the present embodiment, the program for realizing the function of each unit which is stored in the storage medium 1630 is read out to the memory 1620. Then, the processor 1610 executes the program on the memory 1620 so that the above-described processing in each step is performed and the function of each unit is realized.

In FIG. 16, an input interface 1640 is an interface for acquiring information from an external device. In addition, an output interface 1650 is an interface for outputting information to an external device, and is connected to the display unit 120, for example. A bus 1660 is connected to each unit described above so as to enable data exchange.

The detailed description above describes an image processing apparatus, an image processing method and a program. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An image processing apparatus comprising:
image acquisition means for acquiring a tomographic image of a tubular body which is obtained by scanning an inside of a first tubular body using a probe;
detection means for detecting multiple points indicating an inner surface of the tubular body on the tomographic image; and
determination means for determining at least either whether a point indicating the inner surface indicates the first tubular body and whether the point indicates a second tubular body bifurcated from the first tubular body, or whether the point indicating the inner surface indicates a boundary between the first tubular body and the second tubular body, based on a position of the detected multiple points indicating the inner surface.

2. The image processing apparatus according to claim 1, wherein based on a position of a portion where the detected multiple points indicating the inner surface are disconnected in a middle, the determination means determines whether the point indicating the inner surface indicates the boundary between the first tubular body and the second tubular body.

3. The image processing apparatus according to claim 1, wherein based on a distance from an estimated position of a center point of the first tubular body to the point indicating the inner surface, the determination means determines whether the point indicates the first tubular body and whether the point indicates the second tubular body bifurcated from the first tubular body.

4. The image processing apparatus according to claim 3, comprising:
position acquisition means for acquiring the estimated position of the center point of the first tubular body; and
wherein the position acquisition means performs a Hough transform processing on the tomographic image to detect a circle which approximates an inner wall shape of the first tubular body and to acquire a center position of the detected circle as the estimated position of the center point.

5. The image processing apparatus according to claim 3, wherein the determination means determines that the point indicates the first tubular body when a distance between the center point and the point indicating the inner surface is equal to or smaller than a threshold value; and
the determination means determines that the point indicates the second tubular body when the distance between the center point and the point indicating the inner surface is beyond the threshold value.

6. The image processing apparatus according to claim 3, wherein the tomographic image includes an image of a guidewire and a catheter sheath, which are inserted into the first tubular body;
the determination means determines that the point indicates the first tubular body when a distance between the center point and the point indicating the inner surface is equal to or smaller than a threshold value;
the determination means determines that the point indicates a shadow of the guidewire when the distance between the center point and the point indicating the inner surface is beyond the threshold value and when the point is present within a predetermined angular range in a direction from the image of the catheter sheath toward the image of the guidewire on the tomographic image; and
the determination means determines that the point indicates the second tubular body when the distance between the center point and the point indicating the inner surface is beyond the threshold value and when the point is absent within the predetermined angular range in the direction from the image of the catheter sheath toward the image of the guidewire on the tomographic image.

7. The image processing apparatus according to claim 6, wherein the detection means detects an intersection point of a scanning line with a tubular body by performing scanning in each angular direction from the center point;
when the intersection point determined to indicate the first tubular body is present in a first angular direction, when the intersection point determined to indicate the first tubular body is absent in a second angular direction adjacent to the first angular direction, and when the intersection point in the first angular direction is present within a predetermined angular range in a direction from the catheter sheath toward the guidewire, the determination means determines that the intersection point in the first angular direction indicates a boundary between the first tubular body and the shadow of the guidewire; and
when the intersection point in the first angular direction is absent within the predetermined angular range in the direction from the catheter sheath toward the guidewire, the determination means determines that the intersection point in the first angular direction indicates a boundary between the first tubular body and the second tubular body.

8. The image processing apparatus according to claim 6, wherein the detection means detects an intersection point with a tubular body by performing scanning in each angular direction from the center point;
when the intersection point determined to indicate the first tubular body is present in one direction with regard to two adjacent angular directions and when the intersection point determined to indicate the second tubular body is present in the other direction, the determination means determines that the intersection point in one angular direction indicates the boundary between the first tubular body and the second tubular body;
when the intersection point determined to indicate the first tubular body is present in one direction with regard to two adjacent angular directions and when the intersection point determined to indicate the shadow of the guidewire is present in the other direction, the determination means determines that the intersection point in one direction indicates the boundary between the first tubular body and the shadow of the guidewire;
the determination means detects a set of two intersection points which indicate the boundary between the first tubular body and the second tubular body and are present in two different angular directions, that is, a set of intersection points in which all of the intersection points present between the two angular directions indicate the second tubular body, as an intersection point pair indicating the boundary of the second tubular body; and
the determination means detects a set of two intersection points which indicate the boundary between the first tubular body and the shadow of the guidewire and are present in two different angular directions, that is, a set of intersection points in which all of the intersection points present between the two angular directions indicate the shadow of the guidewire, as an intersection point pair indicating the boundary of the shadow of the guidewire.

9. The image processing apparatus according to claim 7, wherein with regard to respective multiple half-lines extending in each angular direction from the center point, the detection means detects a pixel closest to the center point among pixels on the half-line, which have a pixel value within a predetermined range, as the intersection point corresponding to the angular direction.

10. The image processing apparatus according to claim 7, comprising:
determination update means for updating a determination result obtained by the determination means by referring to at least any one of a position of the intersection point indicating the boundary between the first tubular body and the second tubular body and a position of the intersection point indicating the boundary between the first tubular body and the shadow of the guidewire.

11. The image processing apparatus according to claim 10, wherein the determination update means determines that the intersection point determined to indicate the second tubular body by the determination means indicates the first tubular body when a difference in respective distances from the center point to the intersection point pair indicating the boundary of the second tubular body is equal to or greater than a threshold value.

12. The image processing apparatus according to claim 10, wherein the determination update means detects an intersection point between a straight line moved in parallel and a tubular body when the straight line passing through the intersection point pair indicating the boundary of the second tubular body is moved in parallel in a direction away from the center point; and
the determination update means determines that the detected intersection point indicates the second tubular body.

13. The image processing apparatus according to claim 10, wherein the determination update means refers to at least any one of a position of the intersection point indicating the boundary between the first tubular body and the second tubular body and a position of the intersection point indicating the boundary between the first tubular body and the shadow of the guidewire, which are detected from a first tomographic image at a first position of the first tubular body, and updates a determination result obtained by the determination means with regard to a second tomographic image at a second position of the first tubular body.

14. The image processing apparatus according to claim 13, wherein when a distance between the intersection point pair indicating the boundary of the second tubular body which is detected from the first tomographic image is longer than a distance between the intersection point pair indicating the boundary of the second tubular body which is detected from the second tomographic image, and when a distance between the intersection point pair indicating the boundary of the second tubular body which is detected from a third tomographic image at a third position of the first tubular body is longer than the distance between the intersection point pair indicating the boundary of the second tubular body which is detected from the second tomographic image, the determination update means determines that the intersection point determined to indicate the second tubular body by the determination means indicates the first tubular body on the first, second, and third tomographic images; and
the second position is present between the first position and the third position, and the intersection point determined to indicate the second tubular body is present in all of the tomographic images between the first position and the third position.

15. The image processing apparatus according to claim 13, wherein when a difference in two angular directions in which the intersection point pair indicating the boundary of the shadow of the guidewire which is detected from the second tomographic image is present is beyond a threshold value, the determination update means estimates a position of the intersection point pair indicating the boundary of the shadow of the guidewire on the second tomographic image, based on the position of the intersection point pair indicating the boundary of the shadow of the guidewire which is detected from the first tomographic image and the position of the intersection point pair indicating the boundary of the shadow of the guidewire which is detected from the third tomographic image at the third position of the first tubular body;
the determination update means determines that the intersection point absent between the two angular directions in which the estimated intersection point pair indicating the boundary of the shadow of the guidewire is present within the intersection points determined to indicate the shadow of the guidewire by the first determination means indicates the second tubular body on the second tomographic image; and
the second position is present between the first position and the third position.

16. The image processing apparatus according to claim 13, wherein the detection means detects an intersection point with a tubular body when the tubular body proceeds in each angular direction from the center point, in accordance with a first angular direction resolution;
when the intersection point pair indicating the boundary of the second tubular body is detected from the first tomographic image at the first position of the tubular body, and when the intersection point pair indicating the boundary of the second tubular body is not detected from the second tomographic image at the second position adjacent to the first position of the tubular body, with regard to an angular range including a central angle in two angular directions in which the intersection point pair detected from the first tomographic image is present, the determination update means detects the intersection point with the tubular body when the tubular body proceeds in each angular direction from the center point of the second tomographic image, in accordance with a second angular direction resolution which is higher than the first angular direction resolution; and
the determination update means determines whether the detected intersection point indicates the first tubular body and whether the detected intersection point indicates the second tubular body.

17. The image processing apparatus according to claim 1, wherein the first and second tubular bodies are blood vessels.

18. The image processing apparatus according to claim 1, comprising:
display control means for causing display means to display a display screen including at least one of a cross-sectional image, an longitudinal image, and a three-dimensional image of the tubular body; and
wherein in accordance with an input for designating one of multiple bifurcated portions present from the first tubular body to the second tubular body, the display control means updates the display screen to be displayed on the display means so that the display screen includes at least one of the cross-sectional image, the longitudinal image, and the three-dimensional image of the tubular body which includes a designated bifurcated portion.

19. The image processing apparatus according to claim 18, wherein the display control means updates the display screen to be displayed on the display means so that the display screen includes a three-dimensional image of the tubular body which includes the designated bifurcated portion; and
a viewing direction of the three-dimensional image included in the updated display screen is determined according to a bifurcating direction of the second tubular body in the designated bifurcated portion.

20. An image processing method performed by an image processing apparatus, comprising:
an image acquisition step of acquiring a tomographic image of a tubular body which is obtained by scanning an inside of a first tubular body using a probe;
a detection step of detecting multiple points indicating an inner surface of the tubular body on the tomographic image; and
a determination step of determining at least one between whether a point indicating the inner surface indicates the first tubular body and the point indicates a second tubular body bifurcated from the first tubular body, and whether the point indicating the inner surface indicates a boundary between the first tubular body and the second tubular body, based on a position of the detected multiple points indicating the inner surface.

21. A non-transitory computer readable medium containing a computer program having computer readable code embodied to carry out an image processing method performed by an image processing apparatus, the method comprising:
an image acquisition step of acquiring a tomographic image of a tubular body which is obtained by scanning an inside of a first tubular body using a probe;
a detection step of detecting multiple points indicating an inner surface of the tubular body on the tomographic image; and
a determination step of determining at least one between whether a point indicating the inner surface indicates the first tubular body and the point indicates a second tubular body bifurcated from the first tubular body, and whether the point indicating the inner surface indicates a boundary between the first tubular body and the second tubular body, based on a position of the detected multiple points indicating the inner surface.
